(19) United States
(12) Patent Application Publication
(10) Pub. No.: US 2008/0300385 A1
(43) Pub. Date: Dec. 4, 2008

(54) Covalently-linked complexes of HIV Tat and Env proteins

(76) Inventors: Victoria Sharma, Orinda, CA (US); Elaine Kam, Castro Valley, CA (US); Susan W. Barnett, San Francisco, CA (US); Indresh K. Srivastava, Benicia, CA (US)

Correspondence Address: NOVARTIS VACCINES AND DIAGNOSTICS INC., INTELLECTUAL PROPERTY R338, P.O. BOX 8097, Emeryville, CA, 94662-8097, US

(21) Appl. No.: 11/729,076

(22) Filed: Mar. 27, 2007

Related U.S. Application Data
(60) Provisional application No. 60/786,947, filed on Mar. 28, 2006.

(51) Int. Cl. C07K 1/00; C07K 14/00

(52) U.S. Cl. 530/402; 530/350

(57) ABSTRACT

Complexes of HIV Env and Tat proteins are advantageous as immunogens compared to Tat or Env alone, but they may dissociate when combined with a vaccine adjuvant. To avoid dissociation, complexes of Env and Tat are stabilized by the use of covalent cross linking. The extent of cross linking is important to the binding properties of the complexes, and so is controlled to avoid the loss of Env's ability to bind specifically to CD4 and Tat's ability to bind specifically to anti-Tat monoclonal antibodies.

All documents cited herein are incorporated by reference in their entirety.

### RELATED APPLICATIONS

This application claims the benefit of U.S. Provisional Application No. 60/786,947, filed Mar. 28, 2006, which application is incorporated herein in its entirety by reference.

### TECHNICAL FIELD

This invention is in the field of human immunodeficiency virus (HIV) and, in particular, immunogenic protein complexes.

### BACKGROUND OF THE INVENTION

The various proteins encoded within the HIV genome include the envelope glycoprotein (Env) and the trans-activating transcriptional factor (Tat).

In both HIV-1 and HIV-2 the Env protein is initially expressed as a long precursor protein that is subsequently cleaved to give an exterior membrane glycoprotein and a transmembrane glycoprotein. For convenience, these proteins are hereafter referred to by the standard HIV-1 nomenclature i.e. the precursor is ‘gp160’, the membrane glycoprotein is ‘gp120’ and the transmembrane glycoprotein is ‘gp41’. These names are based on approximate molecular weights of the HIV-1 glycoproteins.

The gp120 proteins are on the surface of HIV virions and can interact with the host cell CD4 receptor. This interaction induces a conformational transition in the gp120 protein, leading to the exposure of its ‘V3’ loop. The conformationally-altered gp120 protein can then interact with further host receptors, such as CCR5 and/or CXCR4, as part of the viral entry mechanism. Because of its surface exposure, gp120 has been the main focus of HIV vaccine research over the last 20 years. While anti-Env antibodies that arise during natural infection have been found to neutralize primary HIV isolates, however, the same has not been true of antibodies elicited by Env-based subunit vaccines. Improvements to Env-based vaccines are therefore required.

Tat protein is important in regulating HIV gene expression. Although it is a transcription factor, it has also been found to be released by infected cells and has been proposed as a vaccine antigen.

Reference 1 discloses that Env and Tat proteins can interact to form a complex. The interaction is said to require the presence of the V3 loop in the Env protein. It is proposed that the Tat protein mimics a structural loop of CCR5. The Env and Tat proteins in the complexes may be associated due to their natural affinity, but can be strengthened by forming disulfide bridges or by using protein cross-linking technologies such as the BS3 cross-linker (bis(sulfosuccinimidyl)suberate homobifunctional cross-linker). A vaccine based on a combination of Env and Tat polypeptides is also disclosed in reference 2.

It is an object to provide further and improved complexes of HIV Env and Tat proteins.

### SUMMARY OF THE INVENTION

The present invention is directed to complexes comprising a HIV Env polypeptide and a HIV Tat polypeptide, wherein (i) the Env and Tat polypeptides are covalently linked, and (ii) the complex can bind specifically to CD4. The present invention is also directed to methods for preparing a complex that comprises a HIV Env polypeptide and a HIV Tat polypeptide, comprising the step of allowing Env and Tat polypeptides to interact under reaction conditions where they become covalently linked to each other without removing the Env protein's ability to bind specifically to CD4.

In one embodiment, the Env and Tat of the complexes and methods of the invention are from HIV-1. In certain embodiments, the Env and Tat are from HIV-1 group M. In certain other embodiments, the Env and Tat are from a subtype B strain or from a subtype C strain.

In another embodiment, the Env and Tat are linked via a homobifunctional cross linker. In a further embodiment, the Env and Tat are linked via reaction with formaldehyde or a dialdehyde.

In a particular embodiment, the Env and Tat are present at essentially a 1:1 molar ratio.

### BRIEF DESCRIPTION OF THE DRAWINGS

FIG. 1 shows the results of a Far-Western assay. Lanes are: (1) gp120 and Tat; (2) gp120ΔV2 and Tat; (3) gp140 and Tat; (4) gp140ΔV2 and Tat; (5) gp120 and CD4; and (6) gp120, CD4 and Env.

FIG. 2 shows a quantitative analysis of the results from FIG. 1, measured in arbitrary units.

FIG. 3 shows a Far-Western assay for Env and Tat at four different Env concentrations.

FIG. 4 shows a Far-Western assay for Env and Tat at three different Env:Tat ratios.

FIGS. 5 and 6 show SPR results for Env with Tat (FIG. 5) or CD4 (FIG. 6). The Env proteins were: (A) gp140ΔV2; (B) gp140; (C) gp120ΔV2; and (D) gp120. The X-axes show time (seconds) and the Y-axes shown relative units. The five different lines in each graph are different Env concentrations, with 1000 nM and four serial 2-fold dilutions.

FIG. 7 shows ITC analysis of (A) gp140 and (B) gp140ΔV2 with Tat. In the upper panels, the X-axes show time (minutes) and the Y-axes show μcal/sec. In the lower panels, the X-axes show molar ratios and the Y-axes show kcal/mole of injectant.

FIG. 8 shows a western blot of Env/Tat complexes incubated with different cross-linking reagents under different conditions. Free Tat can be seen towards the bottom of the blots.

FIG. 9 shows western blots of four Env/Tat complexes using (9A) anti-Tat or (9B) anti-Env antibodies. Lanes are: (1) 0.02% glutaraldehyde; (2) 0.04% glutaraldehyde; (3) 0.08% glutaraldehyde; and (4) no cross-linker.

FIG. 10 shows SDS-PAGE analysis of the same complexes. The MW markers in both cases are 15, 25, 30, 35, 50, 75, 105, 160 & 250 kDa.

FIG. 11 shows SEC-HPLC analysis. Plots 1 to 4 match lanes 1 to 4 of FIGS. 8 and 9. Plot 5 is Tat alone, and Plot 6 is Env alone. Plot 7 is SM. The two arrows show Env-bound CD4 (left) and free CD4 (right).

FIGS. 12 and 13 show SPR plots for the same three cross-linked complexes as lanes 1 to 3 of FIGS. 8 and 9, and also gp140ΔV2. The four lines in FIGS. 12A and 13A are, from top to bottom: gp140ΔV2; 0.02%; 0.04%; and 0.08%. The X-axes show time (seconds), and the Y-axes show relative units (RU). FIGS. 12B and 13B show the peak RU value for the four samples, and also for the negative control (buffer only).

FIG. 14 illustrates a general reaction scheme for covalent cross-linking of Env and Tat.

FIG. 15 shows SPR results with Env from a subtype C strain. The y axis shows relative units, and the x axis shows time (seconds). Each line is a different Env concentrations.

### DETAILED DESCRIPTION OF THE INVENTION

Although complexes of Env and Tat proteins are advantageous as immunogens compared to Tat or Env alone, they may dissociate when combined with a vaccine adjuvant. Thus complexes of Env and Tat can be stabilized by the use of covalent cross-linking, but it has been found that the extent of cross-linking is important to the binding properties of the complexes. In particular, too much cross-linking has been found to result in loss of CD4-binding by the Env protein and loss of epitopes by the Tat protein.

Thus the invention provides a complex comprising a HIV Env polypeptide and a HIV Tat polypeptide, wherein (i) the Env and Tat polypeptides are covalently linked, and (ii) the complex can bind specifically to CD4.

The invention also provides a process for preparing a complex that comprises a HIV Env polypeptide and a HIV Tat polypeptide, comprising the step of allowing Env and Tat polypeptides to interact under reaction conditions where they become covalently linked to each other without removing the Env protein's ability to bind specifically to CD4.

The invention also provides a complex comprising a HIV Env polypeptide and a HIV Tat polypeptide, wherein (i) the Env and Tat polypeptides are covalently linked, and (ii) the complex can bind specifically to a monoclonal antibody that specifically binds to HIV Tat protein.

The invention also provides a process for preparing a complex that comprises a HIV Env polypeptide and a HIV Tat polypeptide, comprising the step of allowing Env and Tat polypeptides to interact under reaction conditions where they become covalently linked to each other without removing the Tat protein's ability to bind specifically to an anti-Tat monoclonal antibody.

The invention also provides a complex comprising a HIV Env polypeptide and a HIV Tat polypeptide, wherein (i) the Env and Tat polypeptides are covalently linked, (ii) the complex can bind specifically to CD4, and (iii) the complex can bind specifically to a monoclonal antibody that specifically binds to HIV Tat polypeptide.

The invention also provides a process for preparing a complex that comprises a HIV Env polypeptide and a HIV Tat polypeptide, comprising the step of allowing Env and Tat polypeptides to interact under reaction conditions where they become covalently linked to each other without removing the Env polypeptide's ability to bind specifically to CD4 and without removing the Tat polypeptide's ability to bind specifically to an anti-Tat monoclonal antibody.

### Env/Tat Cross-Linking

The Env and Tat proteins are covalently linked together in the complexes of the invention. Various methods for covalently linking proteins are known in the art e.g. see references 3 & 4. For example, covalent linking may involve the use of homobifunctional cross-linkers, heterobifunctional cross-linkers or zero-length cross-linkers. It may involve reagents directed to sulfhydryl groups in proteins, reagents directed to amino groups in proteins, reagents directed to carboxyl groups in proteins, tyrosine-selective reagents, arginine-specific reagents, histidine-specific reagents, methionine-alkylating reagents, tryptophan-specific reagents, serine-modifying reagents, etc.

A preferred group of cross-linking reagents for use with the invention includes aldehydes, and in particular includes formaldehyde and the dialdehydes. Suitable dialdehydes include glyoxal, malondialdehyde, succinialdehyde, adipaldehyde, α-hydroxyadipaldehyde, glutaraldehyde and phthalaldehyde. Glutaraldehyde and its derivatives are particularly preferred, including 2-methoxy-2,4-dimethylglutaraldehyde, 3-methoxy-2,4-dimethylglutaraldehyde and 3-methylglutar-aldehyde, Pyridoxal phosphates can also be used. Other amino group-directed cross-linkers include bis-imidoesters, bis-succinimidyl derivatives (e.g. bis(sulfosuccinimidyl)suberate, or ‘BS³’), bifunctional aryl halides, bifunctional acylating agents (including di-isocyanates, di-isothiocyanates, bifunctional sulfonyl halides, bis-nitrophenyl esters and bifunctional acylazides), diketones, p-benzoquinone, 2-iminothiolane, erythritolbiscarbonate, mucobromic acid, mucochloric acid, ethylchloroformate and multidiazonium compounds.

Methods for cross-linking proteins using these reagents are known in the art. Generally, the invention will involve mixing Env polypeptide, Tat polypeptide and a linking reagent under conditions that permit the covalent linking reaction to proceed. In some two-step procedures, however, such as those using a heterobifunctional reagent, one of the two polypeptides will be reacted with the linking reagent first, to form an activated polypeptide, and then the activated polypeptide will be reacted with the second polypeptide.

Heterobifunctional linkers with a photoreactive group are also useful. If a linker has one thermoreactive group and one photoreactive group then a first step can involve attachment via the thermoreactive group, and then conjugation to make the complex can be initiated by the use of e.g. UV light. As an alternative, the photoreactive group can be used first.

As mentioned above, the cross-linking reaction is performed to an extent which is not so great as to eliminate critical binding activities of the Env and Tat proteins. Thus the concentration of the Env and Tat proteins, the concentration of the cross-linking reagent(s), the pH, the reaction temperature and the reaction time can be controlled to give the desired degree of cross-linking. When testing a particular combination of Env, Tat and cross-linking reagent then an initial series of reactions can be performed to evaluate suitable reaction conditions.

### The Complex

Complexes of the invention include Env and Tat proteins that are covalently linked. Preferred complexes have essentially a 1:1 molar ratio of Env and Tat. Where the Env is in the form of a trimer, therefore, the preferred complex includes three Tat monomers.

The Env and Tat polypeptides in the complex are preferably from the same type if HIV e.g. both are from HIV-1 or both are from HIV-2. Where the same-HIV types are used, it is also useful to link Env and Tat polypeptides from the same group e.g. within HIV-1, both are from group M, group N or group O. Within group M, it is useful to link Env and Tat polypeptides from the same subtype (or clade) e.g. from subtype A, B, C, D, F, G, H, J or K. It is also possible to use Env or Tat from a CRF (circulating recombinant form) subtype, such as a A/B or A/E CRF. Where a subtype includes sub-subtypes then the Env and Tat polypeptides may be from the same sub-subtype. Using Env and Tat from different groups, subtypes and/or sub-subtypes is not, however, excluded. HIV-1 nomenclature is discussed in more detail in reference 5.

The use of Env and Tat from subtype B or C is preferred. Within a single subtype (or, where applicable, sub-subtype) it is possible to use Env and Tat from the same strain or from different strains. For instance, the Env and Tat polypeptides may both be from the SF162 strain, or the invention may use Env from one strain (e.g. SF162) and Tat from another strain (e.g. BH10).

The Env/Tat complexes of the invention can bind specifically to (a) CD4 and/or (b) a monoclonal antibody that specifically binds to HIV Tat polypeptide. Thus the complexes retain the CD4-binding activity of the uncomplexed Env polypeptide and/or the mAb-binding activity of the uncomplexed Tat polypeptide. Complexes with both of binding activities (a) and (b) are particularly preferred. As mentioned above, retaining these two activities requires an appropriate degree of covalent cross-linking between Env and Tat. Although this degree of cross-linking can vary within a fairly broad range, and thus does not need to be controlled with absolute precision, too little cross-linking leads to unstable complexes and too much cross-linking leads to a loss of binding activity.

Where the complex binds specifically to CD4, this binding activity can be assessed using known assays e.g. as described in reference 6. The assay does not need to use native CD4, however, and it is more typical to use a purified soluble form of CD4 based on its external domain (e.g. see example 5 of ref. 6). The CD4 may also be labeled to facilitate the assay. The CD4 is preferably human CD4. At least 250 SNPs have so far been described for CD4, and any of these polypeptides can be used, such as the REFSEQ CD4 (GI:10835167). The uncomplexed Env will specifically bind to CD4, and this specific binding activity can be retained in the Env/Tat complex. Although the binding activity is not removed, however, the actual binding affinity may change.

Where the complex binds specifically to an anti-Tat monoclonal antibody, a preferred monoclonal antibody is 8D1.8, which is available through the AIDS Research and Reference Reagent Program, Division of AIDS, NIAID, NIH [7]. The use of this antibody in Tat-binding assays has previously been disclosed e.g. in references 8 to 10.

Higher-order oligomers of the Env/Tat complexes have been observed during cross-linking. The invention can use these oligomers, can use Env/Tat complexes that have not formed these oligomers, or can use mixtures of both. If the oligomers are not desired then their formation can be avoided by using appropriate cross-linking conditions, or they can be removed using an appropriate separation technique e.g. a size-based techniques, etc.

### The Env Polypeptide

Complexes of the invention include a HIV Env polypeptide, and various forms of Env polypeptide can be used from HIV-1 or HIV-2. For example, the complex may include a full-length gp160 Env polypeptide, a gp120 Env polypeptide, a gp160 or gp120 polypeptide with one or more deletions, a fusion protein including a gp120 or gp160 polypeptide, etc. Rather than being a full-length Env precursor, however, the invention will typically use a shortened protein.

The amino acid sequence of the full-length HIV-1 Env precursor from the REFSEQ database (GI:9629363) is a 856mer shown below (SEQ ID NO: 1 herein):

MRVKEKYQHLWRWGWRWGTMLLGMLMICSATEKLWVTVYYGVPVWKEATT
TLFCASDAKAYDTEVHNVWATHACVPTDPNPQEVVLVNVTENFNMWKNDM
VEQMHEDIISLWDQSLKPCVKLTPLCVSLKCTDLKNDTNTNSSSGRMIME
KGEIKNCSFNISTSIRGKVQKEYAFFYKLDIIPIDNDTTSYKLTSCNTSV
ITQACPKVSFEPIPIHYCAPAGFAILKCNNKTFNGTGPCTNVSTVQCTHG
IRPVVSTQLLLNGSLAEEEVVIRSVNFTDNAKTIIVQLNTSVEINCTRPN
NNTRKRIRIQRGPGRAFVTIGKIGNMRQAHCNISRAKWNNTLKQIASKLR
EQFGNNKTIIFKQSSGGDPEIVTHSFNCGGEFFYCNSTQLFNSTWFNSTW
STEGSNNTEGSDTITLPCRIKQIINNWQKVGKAMYAPPISGQIRCSSNIT
GLLLTRDGGNSNNESEIFRPGGGDMRDNWRSELYKYKVVKIEPLGVAPTK
AKRRVVQREKRAVGIGALFLGFLGAAGSTMGAASMTLTVQARQLLSGIVQ
QQNNLLRAIEAQQHLLQLTVWGIKQLQARILAVERYLKDQQLLGIWGCSG
KLICTTAVPWNASWSNKSLEQIWNHTTWMEWDREINNYTSLIHSLIEESQ
NQQEKNEQELLELDKWASLWNWFNITNWLWYIKLFIMIVGGLVGLRIVFA
VLSIVNRVRQGYSPLSFQTHLPTPRGPDRPEGIEEEGGERDRDRSIRLVN
GSLALIWDDLRSLCLFSYHRLRDLLLIVTRIVELLGRRGWEALKYWWNLL
QYWSQELKNSAVSLLNATAIAVAEGTDRVIEVVQGACRAIRHIPRRIRQG
LERILL

The wild-type HIV-1 precursor protein is cleaved to give the surface glycoprotein gp120 (e.g. amino acids 29-511 of SEQ ID NO: 1; SEQ ID NO: 2 herein) and the transmembrane domain gp41 (e.g. amino acids 512-856 of SEQ ID NO: 1; SEQ ID NO: 3 herein):

MRVKEKYQHLWRWGWRWGTMLLGMLMIC/SATEKLWVTVYYGVPVWKEAT
TTLFCASDAKAYDTEVHNVWATHACVPTDPNPQEVVLVNVTENFNMWKND
MVEQMHEDIISLWDQSLKPCVKLTPLCVSLKCTDLKNDTNTNSSSGRMIM
EKGEIKNCSFNISTSIRGKVQKEYAFFYKLDIIPIDNDTTSYKLTSCNTS
VITQACPKVSFEPIPIHYCAPAGFAILKCNNKTFNGTGPCTNVSTVQCTH
GIRPVVSTQLLLWGSLAEEEVVIRSVNFTDNAKTIIVQLNTSVEINCTRP
NNNTRKRIRIQRGPGRAFVTIGKIGNMRQAHCNISRAKWNNTLKQIASKL
REQFGNNKTIIFKQSSGGDPEIVTHSFNCGGEFFYCNSTQLFNSTWFNST
WSTEGSNNTEGSDTITLPCRIKQIINNWQKVGKAMYAPPISGQIRCSSNI
TGLLLTRDGGNSNNESEIFRPGGGDMRDNWRSELYKYKVVKIEPLGVAPT
KAKRRVVQREKR/AVGIGALFLGFLGAAGSTMGAASMTLTVQARQLLSGI
VQQQNNLLRAIEAQQHLLQLTVWGIKQLQARILAVERYLKDQQLLGIWGC
SGKLICTTAVPWNASWSNKSLEQIWNHTTWMEWDREINNYTSLIHSLIEE
SQNQQEKNEQELLELDKWASLWNWFNITNWLWYIKLFIMIVGGLVGLRIV
FAVLSIVNRVRQGYSPLSFQTHLPTPRGPDRPEGIEEEGGERDRDRSIRL
VNGSLALIWDDLRSLCLFSYHRLRDLLLIVTRIVELLGRRGWEALKYWWN
LLQYWSQELKNSAVSLLNATAIAVAEGTDRVIEVVQGACRAIRHIPRRIR
QGLERILL

The hypervariable regions within the gp120 region are located as follows, numbered according to SEQ ID NO: 1: V1=131-157; V2=157-196; V3=296-331; V4=385-418; and V5=461-471. Within the overall C1-V1-V2-C2-V3-C3-V4-C4-V5-C5 arrangement of gp120, therefore, the subdomains are as follows (numbered according to SEQ ID NO: 2): 1-102; 103-129; 129-168; 169-267; 268-303; 304-356; 357-390; 391-432; 433-443; and 444-483. Residues that have been identified as important for CD4 binding include (numbered according to SEQ ID NO: 1) Asp-368, Glu-370, Trp-427, Val-430 and Pro-438, and the immunodominant region is residues 588-607. These features can be identified in other HIV-1 Env sequences by performing a suitable sequence alignment. Pre-aligned sequences from numerous strains, annotated with these features, can also be found in the Los Alamos HIV Sequence Compendia [1,1].

The amino acid sequence of a full-length HIV-2 Env precursor (GI:2144996) is a 852mer shown below (SEQ ID NO: 4 herein):

MCGKSLLCVASLLASAYLVYCTQYVTVFYGVPVWRNASIPLFCATKNRDT
WGTIQCKPDNDDYQEITLNVTEAFDAWDNTVTEQAVEDVWSLFETSIKPC
VKLTPLCVAMSCNSTTNNTTTTGSTTGMSEINETSPSYSDNCTGLGKEEI
VNCQFYMTGLERDKKKQYNETWYSKDVVCESNNTKDGKNRCYMNHCNTSV
ITESCDKHYWDAIKFRYCAPPGYALLRCNDTNYSGFEPKCSKVVASTCTR
MMETQTSTWFGFNGTPAENRTYIYWHGRDNRTIISLNKYYNLSIHCKRPG
NKTVVPITLMSGLVFHSQPINTRPRQAWCWFKGKWREAMQEVKQTLIKHP
RYKGTNDTKNINFTKPGRGSDPEVAYMWTNCRGEFLYCNMTWFLNWVENR
PNQTQHNYAPCHIRQIINTWHKVGKNVYLPPREGQLTCNSTVTSIIANID
VNSNQTNITFSAEVAELYRLELGDYKLIEVTPIGFAPTREKRYSSAPVRN
KRGVFVLGFLGFLATAGSAMGAASLTLSAQSRTLLAGIVQQQQQLLDVVK
RQQEMLRLTVWGTKNLQARVTAIEKYLKDQAQLNSWGCAFRQVCHTTVPW
VNDSLSPDWNNMTWQEWEKQVRYLEANISQSLEQAQIQQEKNMYELQKLN
SWDVFGNWFDLTSWIKYIQYGVYIVVGVIVLRIAIYIVQLLSRLRKGYRP
VFSSPPGYLQQIHIHTDRGQPANEGTEEDDRDDDGYDLXPWPINYIHFLI
HLLTRLLTGLYKICRDLLSTNSPTHRLISQNLTAIRDWLRLKAAYLQYGG
EWIQEAFQAFAKTTRETLASAWGGLCAAVQRVGRGILAVPRRIRQGAEIA
LL

The HIV-2 Env precursor protein is cleaved to give the surface glycoprotein (e.g. amino acids 20-502 of SEQ ID NO: 4; SEQ ID NO: 5 herein) and the transmembrane domain (e.g. amino acids 503-852 of SEQ ID NO: 4; SEQ ID NO: 6 herein):

MCGKSLLCVASLLASAYLV/YCTQYVTVFYGVPVWRNASIPLFCATKNRD
TWGTIQCKPDNDDYQEITLNVTEAFDAWDNTVTEQAVEDVWSLFETSIKP
CVKLTPLCVAMSCNSTTNNTTTTGSTTGMSEINETSPSYSDNCTGLGKEE
IVNCQFYMTGLERDKKKQYNETWYSKDVVCESNNTKDGKNRCYMNHCNTS
VITESCDKHYWDAIKFRYCAPPGYALLRCNDTNYSGFEPKCSKVVASTCT
RMMETQTSTWFGFNGTRAENRTYIYWHGRDNRTIISLNKYYNLSIHCKRP
GNKTVVPITLMSGLVFHSQPINTRPRQAWCWFKGKWREAMQEVKQTLIKH
PRYKGTNDTKNINFTKPGRGSDPEVAYMWTNCRGEFLYCNMTWFLNWVEN
RPNQTQHNYAPCHIRQIINTWHKVGKNVYLPPREGQLTCNSTVTSIIANI
DVNSNQTNITFSAEVAELYRLELGDYKLIEVTPIGFAPTREKRYSSAPVR
NKR/GVFVLGFLGFLATAGSAMGAASLTLSAQSRTLLAGIVQQQQQLLDV
VKRQQEMLRLTVWGTKNLQARVTAIEKYLKDQAQLNSWGCAFRQVCHTTV
PWVNDSLSPDWNNMTWQEWEKQVRYLEANISQSLEQAQIQQEKNMYELQK
LNSWDVFGNWFDLTSWIKYIQYGVYIVVGVIVLRIAIYIVQLLSRLRKGY
RPVFSSPPGYLQQIHIHTDRGQPANEGTEEDDRDDDGYDLXPWPINYIHF
LIHLLTRLLTGLYKICRDLLSTNSPTHRLISQNLTAIRDWLRLKAAYLQY
GGEWIQEAFQAFAKTTRETLASAWGGLCAAVQRVGRGILAVPRRIRQGAE
IALL

The hypervariable regions etc. can, again, be identified by sequence alignment and by reference to the alignments in the Los Alamos HIV Sequence Compendia. For example, the V3 loop is at Cys-296 to Cys-329.

Other specific Env sequences that can be used include those disclosed in references 12 to 16

As mentioned above, the invention will typically use a shortened Env polypeptide. The shortening will involve the removal of one of more amino acids from the full-length sequence e.g. truncation at the C-terminus and/or N-terminus, deletion of internal residues, removal of subdomains [17], and combinations of these approaches.

For instance, it is known to make a soluble form of the Env precursor by removing its transmembrane domain and cytoplasmic tail. This polypeptide, which includes the gp120 sequence and the ectodomain of gp41, is known as ‘gp140’ [18], and has been reported to be a better immunogen than gp120 [19]. Thus the precursor is truncated at its C-terminus e.g. after Lys-665 of SEQ ID NO:1, giving a mature gp140 sequence of a 637mer (SEQ ID NO:7 herein) having amino acids Ser-29 to Lys-665 of SEQ ID NO: 1. Thus the Env polypeptide of the invention may include a portion of gp41 but not include its transmembrane domain.

It is also known to make deletions within the V2 loop of the Env precursor, to give ‘ΔV2’ mutants. For instance, one or more amino acids within the 40-mer V2 loop can be deleted (e.g. at least 2, 3, 4, 5, 6, 7, 8, 9, 10, 12, 14, 16, 18, 20, 22, 24, 26, 28, 30, 32, 34, 36, 38 or more amino acids). Deletions within the V2 loop have been reported to improve immunogenicity of Env polypeptides [20,21]. Env polypeptides with deletions and/or substitutions in the V2 loop are preferred with the present invention, as these have been found to be particularly useful in forming Env/Tat complexes. In particular, Env/Tat complexes are not seen with monomeric gp120 unless its V2 loop is mutated. Amino acids deleted from the V2 loop may be substituted with other amino acids e.g. it is known to replace the central portion of the V2 loop with a Gly-Ala-Gly tripeptide. For example, a ΔV2 mutant may have the following sequence (SEQ ID NO: 8):

SATEKLWVTVYYGVPVWKEATTTLFCASDAKAYDTEVHNVWATHACVPTD
PNPQEVVLVNVTENFNMWKNDMVEQMHEDIISLWDQSLKPCVKLTPLCVS
LKCTDLKNDTNTNSSSGRMIMEKGEIKNCXCNTSVITQACPKVSFEPIPI
HYCAPAGFAILKCNNKTFNGTGPCTNVSTVQCTHGIRPVVSTQLLLNGSL
AEEEVVIRSVNFTDNAKTIIVQLNTSVEINCTRPNNNTRKRIRIQRGPGR
AFVTIGKIGNMRQAHCNISRAKWNNTLKQIASKLREQFGNNKTIIFKQSS
GGDPEIVTHSFNCGGEFFYCNSTQLFNSTWFNSTWSTEGSNNTEGSDTIT
LPCRIKQIINMWQKVGKAMYAPPISGQIRCSSNITGLLLTRDGGNSNNES
EIFRPGGGDMRDNWRSELYKYKVVKIEPLGVAPTKAKRRVVQREKR

where the ‘X’ at position 130 represents a mutant V2 loop e.g. with between 4 and 15 amino acids.

A particularly preferred Env polypeptide for use with the invention is a gp140 protein with a ΔV2 mutation from HIV-1 strain SF162. In its mature form, after cleavage of a signal sequence and secretion (see FIG. 24 of reference 12), this polypeptide has the following amino acid sequence (SEQ ID NO: 9):

SAVEKLWVTVYYGVPVWKEATTTLFCASDAKAYDTEVHNVWATHACVPTD
PNPQEIVLENVTENFNMWKNNMVEQMHEDIISLWDQSLKPCVKLTPLCVT
LHCTNLKNATNTKSSNWKEMDRGEIKNCSFKVGAGKLINCNTSVITQACP
KVSFEPIPIHYCAPAGFAILKCNDKKFNGSGPCTNVSTVQCTHGIRPVVS
TQLLLNGSLAEEGVVIRSENFTDNAKTIIVQLKESVEINCTRPNNNTRKS
ITIGPGRAFYATGDIIGDIRQAHCNISGEKWNNTLKQIVTKLQAQFGNKT
IVFKQSSGGDPEIVMHSFNCGGEFFYCNSTQLFNSTWNNTIGPNNTNGTI
TLPCRIKQIINRWQEVGKAMYAPPIRGQIRCSSNITGLLLTRDGGKEISN
TTEIFRPGGGDMRDNWRSELYKYKVVKIEPLGVAPTKAISSVVQSEKSAV
TLGAMFLGFLGAAGSTMGARSLTLTVQARQLLSGIVQQQNNLLRAIEAQQ
HLLQLTVWGIKQLQARVLAVERYLKDQQLLGIWGCSGKLICTTAVPWNAS
WSNKSLDQIWNNMTWMEWEREIDNYTNLIYTLIEESQNQQEKNEQELLEL
DKWASLWNWFDISKWLWYI

As the HIV genome is in a state of constant flux, and contains several domains that exhibit relatively high degrees of variability between isolates, the invention is not limited to the use of Env polypeptides having the exact sequence of a known HIV polypeptide. Thus the Env polypeptide used according to the invention may be selected from:
(i) a polypeptide comprising an amino acid sequence selected from SEQ ID NOs: 1, 2, 4, 5, 7, 8 and 9;
(ii) a polypeptide comprising an amino acid sequence that has sequence identity to an amino acid sequence selected from SEQ ID NOs: 1, 2, 4, 5, 7, 8 and 9;
(iii) a polypeptide comprising an amino acid sequence that, compared to an amino acid sequence selected from SEQ ID NOs: 1, 2, 4, 5, 7, 8 and 9, has one or more (e.g. 1, 2, 3, 4, 5, 6, 7, 8, 9, 10, etc.) substitutions and/or deletions and/or insertions;
(iv) a polypeptide comprising an amino acid sequence comprising a fragment of at least n consecutive amino acids from an amino acid sequence selected from SEQ ID NOs: 1, 2, 4, 5, 7, 8 and 9, where n is 7 or more; or
(v) a polypeptide comprising a sequence of p amino acids that, when aligned with an amino acid sequence selected from SEQ ID NOs: 1, 2, 4, 5, 7, 8 and 9 using a pairwise alignment algorithm, has at least x·y identical aligned monomers in each window of x amino acids moving from N-terminus to C-terminus, where: p>x; there are p−x+1 windows; x is selected from 20, 25, 30, 35, 40, 45, 50, 60, 70, 80, 90, 100, 150, 200, 250, 300, 350, 400, 450, 500, 550, 600, 650, 700, 750, 800 or 850; y is selected from 0.50, 0.60, 0.70, 0.75, 0.80, 0.85, 0.90, 0.91, 0.92, 0.93, 0.94, 0.95, 0.96, 0.97, 0.98, or 0.99; and, if x·y is not an integer, it is rounded up to the nearest integer.

These polypeptides include homologs, orthologs, allelic variants and mutants of SEQ ID NOs 1, 2, 4, 5, 7, 8 and 9. For instance, it is known to mutate natural Env sequences to improve resistance to proteases. The polypeptides also include fusion polypeptides, in which the Env sequence is fused to non-Env sequence. For instance, it is known to fuse Env sequences without the native leader peptide to leader peptides from non-Env proteins e.g. from tissue plasminogen activator.

Within category (ii), the degree of sequence identity may be greater than 50% (e.g. 60%, 70%, 75%, 80%, 85%, 90%, 91%, 92%, 93%, 94%, 95%, 96%, 97%, 98%, 99% or more). Identity between polypeptides is preferably determined by the Smith-Waterman homology search algorithm as implemented in the MPSRCH program (Oxford Molecular), using an affine gap search with parameters gap open penalty=12 and gap extension penalty=1.

Within category (iii), each substitution involves a single amino acid, each deletion preferably involves a single amino acid, and each insertion preferably involves a single amino acid. These changes may arise deliberately (e.g. by site-directed mutagenesis) or naturally (e.g. through virus evolution or through spontaneous mutation). The polypeptides in category (iii) may have one or more (e.g. 1, 2, 3, 4, 5, 6, 7, 8, 9, 10, etc.) single amino acid substitutions relative to SEQ ID NO: 1, 2, 4, 5, 7, 8 or 9. These polypeptides may have one or more (e.g. 1, 2, 3, 4, 5, 6, 7, 8, 9, 10, etc.) single amino acid deletions relative to SEQ ID NO: 1, 2, 4, 5, 7, 8 or 9. These polypeptide s may have one or more (e.g. 1, 2, 3, 4, 5, 6, 7, 8, 9, 10, etc.) single amino acid insertion relative to SEQ ID NO: 1, 2, 4, 5, 7, 8 or 9. The substitutions, insertions and/or deletions may be at separate locations or may be contiguous. Substitutions may be conservative i.e. replacements of one amino acid with another which has a related side chain. Genetically-encoded amino acids are generally divided into four families: (1) acidic i.e. aspartate, glutamate; (2) basic i.e. lysine, arginine, histidine; (3) non-polar i.e. alanine, valine, leucine, isoleucine, proline, phenylalanine, methionine, tryptophan; and (4) uncharged polar i.e. glycine, asparagine, glutamine, cysteine, serine, threonine, tyrosine. Phenylalanine, tryptophan, and tyrosine are sometimes classified jointly as aromatic amino acids. In general, substitution of single amino acids within these families does not have a major effect on the biological activity. Various substitutions have been described for use with Env polypeptides e.g. it is known to inactivate the cleavage site between gp120 and gp41 (e.g. by a Lys→Ser substitution) in order to provide a polypeptide that remains in full-length form, or to remove the ‘clipping’ site in the V3 loop [22], or to delete or substitute glycosylation sites, particularly N-glycosylation sites (i.e. asparagine residues).

Within category (iv), the value of n may be greater than 7 e.g. 8, 10, 12, 14, 16, 18, 20, 22, 24, 26, 28, 30, 35, 40, 45, 50, 60, 70, 80, 90, 100, 150, 200, 250, 300, 350, 400, 450, 500, 550, 600, 650, 700, 750, 800, 850 or more. The fragment may comprise at least one T-cell and/or B-cell epitope of the sequence. T- and B-cell epitopes can be identified empirically (e.g. using PEPSCAN [23,24] or similar methods), or they can be predicted (e.g. using the Jameson-Wolf antigenic index [25], matrix-based approaches [26], TEPITOPE [27], neural networks [28], OptiMer & EpiMer [29,30], ADEPT [31], Tsites [32], hydrophilicity [33], antigenic index [34] or the methods disclosed in ref. 35, etc.).

Within category (v), the preferred pairwise alignment algorithm is the Needleman-Wunsch global alignment algorithm [36], using default parameters (e.g. with Gap opening penalty=10.0, and with Gap extension penalty=0.5, using the EBLOSUM62 scoring matrix). This algorithm is conveniently implemented in the needle tool in the EMBOSS package [37].

Env polypeptide is found in oligomeric form on the HIV virion, and preferred Env polypeptides used with the invention can also form oligomers, and in particular trimers. For instance, ΔV2 mutants of gp140 have been shown to form trimers [20]. As described below, Env/Tat complexes are not formed using monomeric gp120, unless its V2 loop is mutated, but are formed from trimeric gp140 without requiring any V2 mutation.

Within this group of Env polypeptides that may be used with the invention, a preferred feature is that the polypeptide should retain the ability of natural Env to bind to CD4. Where an Env/Tat complex of the invention can bind specifically to CD4 then the Env component of the complex can itself bind to CD4 even in the absence of Tat. When making the complex, for instance, a CD4-binding Env polypeptide will be mixed with a Tat polypeptide, and CD4-binding activity is not removed by complex formation, although the actual binding affinity may change.

### The Tat Polypeptide

Complexes of the invention include a HIV Tat polypeptide, and various forms of Tat polypeptide can be used from HIV-1 or HIV-2. The length of the Tat polypeptide varies depending on virus strain. The amino acid sequence of the full-length HIV-1 Tat polypeptide from the REFSEQ database (GI:9629358) is a 86mer shown below (SEQ ID NO: 10 herein):

MEPVDPRLEPWKHPGSQPKTACTNCYCKKCCFHCQVCFITKALGISYGRK
KRRQRRRAHQNSQTHQASLSKQPTSQPRGDPTGPKE

Within the various HIV-1 Tat polypeptide sequences, Cys-22 and Cys-37 are conserved and form an intramolecular disulfide bond. The RKKRRQRRR 9-mer is a nuclear localization signal. These features can be identified in other HIV-1 Env sequences by performing a suitable sequence alignment. Pre-aligned sequences from numerous strains, annotated with these features, can also be found in the Los Alamos HIV Sequence Compendia [11].

The amino acid sequence of a full-length HIV-2 Tat polypeptide (GI:41056781) is a 130mer shown below (SEQ ID NO: 11 herein):

METPLKAPESSLMSYNEPSSCTSERDVGSQELAKQGEELLSQLHRPLEPC
NNKCYCKGCCFHCQLCFLNKGLGICYDRKGRRRRTPKKTKAHSSSASDKS
ISTRTGNSQPEKKQKKTLETTLETARGLGR

An alignment of this and other HIV-2 Tat sequences can be found in the Los Alamos HIV Sequence Compendia.

Other specific tat sequences that can be used include those disclosed in references 12-15 & 38.

A particularly preferred Tat polypeptide for use with the invention is from HIV-1 strain BH10. This polypeptide has the following amino acid sequence (SEQ ID NO: 12; GI:62291022):

MEPVDPRLEPWKHPGSQPKTACTNCYCKKCCFHCQVCFITKALGISYGRK
KRRQRRRPPQGSQTHQVSLSKQPTSQSRGDPTGPKE

As the HIV genome is in a state of constant flux, and contains several domains that exhibit relatively high degrees of variability between isolates, the invention is not limited to the use of Tat polypeptides having the exact sequence of a known HIV polypeptide. Thus the Tat polypeptide used according to the invention may be selected from:
(i) a polypeptide comprising an amino acid sequence selected from SEQ ID NOs: 10, 11 and 12;
(ii) a polypeptide comprising an amino acid sequence that has sequence identity to an amino acid sequence selected from SEQ ID NOs: 10, 11 and 12;
(iii) a polypeptide comprising an amino acid sequence that, compared to an amino acid sequence selected from SEQ ID NOs: 10, 11 and 12, has one or more (e.g. 1, 2, 3, 4, 5, 6, 7, 8, 9, 10, etc.) substitutions and/or deletions and/or insertions;
(iv) a polypeptide comprising an amino acid sequence comprising a fragment of at least n consecutive amino acids from an amino acid sequence selected from SEQ ID NOs: 10, 11 and 12, where n is 7 or more; or
(v) a polypeptide comprising a sequence of p amino acids that, when aligned with an amino acid sequence selected from SEQ ID NOs: 10, 11 and 12 using a pairwise alignment algorithm, has at least x·y identical aligned monomers in each window of x amino acids moving from N-terminus to C-terminus, where: p>x; there are p−x+1 windows; x is selected from 20, 25, 30, 35, 40, 45, 50, 55, 60, 65, 70, 75, 80 or 85; y is selected from 0.50, 0.60, 0.70, 0.75, 0.80, 0.85, 0.90, 0.91, 0.92, 0.93, 0.94, 0.95, 0.96, 0.97, 0.98, or 0.99; and, if x·y is not an integer, it is rounded up to the nearest integer.

These polypeptides include homologs, orthologs, allelic variants and mutants of SEQ ID NOs 10, 11 and 12. They also include fusion polypeptides, in which the Tat sequence is fused to non-Tat sequence.

Within category (ii), the degree of sequence identity may be greater than 50% (e.g. 60%, 70%, 75%, 80%, 85%, 90%, 91%, 92%, 93%, 94%, 95%, 96%, 97%, 98%, 99% or more). Identity between polypeptides is preferably determined by the Smith-Waterman homology search algorithm as implemented in the MPSRCH program (Oxford Molecular), using an affine gap search with parameters gap open penalty=12 and gap extension penalty=1.

Within category (iii), each substitution involves a single amino acid, each deletion preferably involves a single amino acid, and each insertion preferably involves a single amino acid. These changes may arise deliberately (e.g. by site-directed mutagenesis) or naturally (e.g. through virus evolution or through spontaneous mutation). The polypeptides in category (iii) may have one or more (e.g. 1, 2, 3, 4, 5, 6, 7, 8, 9, 10, etc.) single amino acid substitutions relative to SEQ ID NO: 10, 11 or 12. These polypeptides may have one or more (e.g. 1, 2, 3, 4, 5, 6, 7, 8, 9, 10, etc.) single amino acid deletions relative to SEQ ID NO: 10, 11 or 12. These polypeptide s may have one or more (e.g. 1, 2, 3, 4, 5, 6, 7, 8, 9, 10, etc.) single amino acid insertion relative to SEQ ID NO: 10, 11 or 12. The substitutions, insertions and/or deletions may be at separate locations or may be contiguous. As mentioned above, substitutions may be conservative.

Within category (iv), the value of n may be greater than 7 e.g. 8, 10, 12, 14, 16, 18, 20, 22, 24, 26, 28, 30, 35, 40, 45, 50, 60, 70, 80, 90, 100, 150, 200, 250, 300, 350, 400, 450, 500, 550, 600, 650, 700, 750, 800, 850 or more. The fragment may comprise at least one T-cell and/or B-cell epitope of the sequence. As described above, such epitopes can be identified empirically or can be predicted.

Within category (v), the preferred pairwise alignment algorithm is the Needleman-Wunsch global alignment algorithm as described above.

### Pharmaceutical Compositions

Complexes of the invention can be used as the active ingredient in immunogenic compositions. These compositions can be administered to animals in order to elicit an immune response. The immune response preferably includes a humoral (e.g. an antibody response, such as a neutralizing antibody response) and/or a cellular response against Env and/or Tat. In a patient already infected with HIV, the immune response may reduce the severity of the infection (e.g. reduce viral load) and may even result in clearance of HIV infection. In a patient who is not infected with HIV, the immune response may reduce the risk of future HIV infection and may even be protective against future HIV infection. These effects arising from administration of the immunogenic composition of may be augmented by, or also require, the use of other anti-HIV strategies e.g. the administration of antivirals, including but not limited to nucleoside reverse transcriptase inhibitors, non-nucleoside reverse transcriptase inhibitors, protease inhibitors, entry inhibitors, fusion inhibitors, etc.

Immunogenic compositions will include an immunologically effective amount of the complex. By ‘immunologically effective amount’, it is meant that the administration of that amount to an individual, either in a single dose or as part of a series, is effective for the desired treatment or prevention. This amount can vary depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials, and a typical quantity of complex per dose is between 1 μg and 10 mg per antigen.

Immunogenic compositions of the invention are pharmaceutically acceptable. They usually include components in addition to the complexes e.g. they typically include one or more pharmaceutical carrier(s) and/or excipient(s). A thorough discussion of such components is available in reference 39.

Compositions will generally be in aqueous form.

To control tonicity, it is preferred to include a physiological salt, such as a sodium salt. Sodium chloride (NaCl) is preferred, which may be present at between 1 and 20 mg/ml. Other salts that may be present include potassium chloride, potassium dihydrogen phosphate, disodium phosphate dehydrate, magnesium chloride, calcium chloride, etc.

Compositions will generally have an osmolality of between 200 mOsm/kg and 400 mOsm/kg, preferably between 240-360 mOsm/kg, and will more preferably fall within the range of 290-310 mOsm/kg.

Compositions may include one or more buffers. Typical buffers include: a phosphate buffer; a Tris buffer; a borate buffer; a succinate buffer; a histidine buffer; or a citrate buffer. Buffers will typically be included in the 5-20 mM range.

The pH of a composition will generally be between 5 and 8, and more typically between 6 and 7.

The composition is preferably sterile. The composition is preferably non-pyrogenic e.g. containing <1 EU (endotoxin unit, a standard measure) per dose, and preferably <0.1 EU per dose. The composition is preferably gluten free.

Compositions of the invention may include detergent e.g. a polyoxyethylene sorbitan ester surfactant (known as ‘Tweens’), an octoxynol (such as octoxynol-9 (Triton X-100) or t-octylphenoxypolyethoxyethanol), etc.

Vaccines may be administered in a dosage volume of about 0.5 ml.

### Vaccine Adjuvants

Compositions of the invention may advantageously include an adjuvant, which can function to enhance the immune responses (humoral and/or cellular) elicited in a patient who receives the composition. Adjuvants that can be used with the invention include, but are not limited to:
A mineral-containing composition, including calcium salts and aluminum salts (or mixtures thereof). Calcium salts include calcium phosphate (e.g. the “CAP” particles disclosed in ref. 40). Aluminum salts include hydroxides, phosphates, sulfates, etc., with the salts taking any suitable form (e.g. gel, crystalline, amorphous, etc.). Adsorption to these salts is preferred. The mineral containing compositions may also be formulated as a particle of metal salt [41]. Aluminum salt adjuvants are described in more detail below.
An oil-in-water emulsion, as described in more detail below.
An immunostimulatory oligonucleotide, such as one containing a CpG motif (a dinucleotide sequence containing an unmethylated cytosine linked by a phosphate bond to a guanosine), a TpG motif [42], a double-stranded RNA, an oligonucleotide containing a palindromic sequence, or an oligonucleotide containing a poly(dG) sequence. Immunostimulatory oligonucleotides can include nucleotide modifications/analogs such as phosphorothioate modifications and can be double-stranded or (except for RNA) single-stranded. References 43 to 45 disclose possible analog substitutions e.g. replacement of guanosine with 2′-deoxy-7-deazaguanosine. The adjuvant effect of CpG oligonucleotides is further discussed in refs. 46-51. A CpG sequence may be directed to TLR9, such as the motif GTCGTT or TTCGTT [52]. The CpG sequence may be specific for inducing a Th1 immune response, such as a CpG-A ODN (oligodeoxynucleotide), or it may be more specific for inducing a B cell response, such a CpG-B ODN. CpG-A and CpG-B ODNs are discussed in refs. 53-55. Preferably, the CpG is a CpG-A ODN. Preferably, the CpG oligonucleotide is constructed so that the 5′ end is accessible for receptor recognition. Optionally, two CpG oligonucleotide sequences may be attached at their 3′ ends to form “immunomers”. See, for example, references 52 & 56-58. A useful CpG adjuvant is CpG7909, also known as ProMune™ (Coley Pharmaceutical Group, Inc.). Immunostimulatory oligonucleotides will typically comprise at least 20 nucleotides. They may comprise fewer than 100 nucleotides.
3-O-deacylated monophosphoryl lipid A (‘3dMPL’, also known as ‘MPL™’) [59-62]. 3dMPL has been prepared from a heptoseless mutant of Salmonella minnesota, and is chemically similar to lipid A but lacks an acid-labile phosphoryl group and a base-labile acyl group. Preparation of 3dMPL was originally described in reference 63. 3dMPL can take the form of a mixture of related molecules, varying by their acylation (e.g. having 3, 4, 5 or 6 acyl chains, which may be of different lengths). The two glucosamine (also known as 2-deoxy-2-amino-glucose) monosaccharides are N-acylated at their 2-position carbons (i.e. at positions 2 and 2′), and there is also O-acylation at the 3′ position.
An imidazoquinoline compound, such as Imiquimod (“R-837”) [64,65], Resiquimod (“R-848”) [66], and their analogs; and salts thereof (e.g. the hydrochloride salts). Further details about immunostimulatory imidazoquinolines can be found in references 67 to 71.
A thiosemicarbazone compound, such as those disclosed in reference 72. Methods of formulating, manufacturing, and screening for active compounds are also described in reference 72. The thiosemicarbazones are particularly effective in the stimulation of human peripheral blood mononuclear cells for the production of cytokines, such as TNF-α.
A tryptanthrin compound, such as those disclosed in reference 73. Methods of formulating, manufacturing, and screening for active compounds are also described in reference 73. The thiosemicarbazones are particularly effective in the stimulation of human peripheral blood mononuclear cells for the production of cytokines, such as TNF-α.
A nucleoside analog, such as: (a) Isatorabine (ANA-245; 7-thia-8-oxoguanosine):

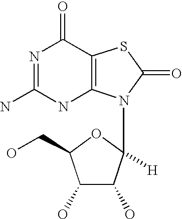

and prodrugs thereof; (b) ANA975; (c) ANA-025-1; (d) ANA380; (e) the compounds disclosed in references 74 to 76; (f) a compound having the formula:

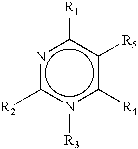

wherein:
   R₁ and R₂ are each independently H, halo, —NRₐR_{b}, —OH, C₁₋₆ alkoxy, substituted C₁₋₆ alkoxy, heterocyclyl, substituted heterocyclyl, C₆₋₁₀ aryl, substituted C₆₋₁₀ aryl, C₁₋₆ alkyl, or substituted C₁₋₆ alkyl;
   R₃ is absent, H, C₁₋₆ alkyl, substituted C₁₋₆ alkyl, C₆₋₁₀ aryl, substituted C₆₋₁₀ aryl, heterocyclyl, or substituted heterocyclyl;
   R₄ and R₅ are each independently H, halo, heterocyclyl, substituted heterocyclyl, —C(O)—R_{d}, C₁₋₆ alkyl, substituted C₁₋₆ alkyl, or bound together to form a 5 membered ring as in R₄₋₅:

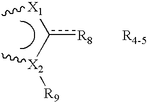

the binding being achieved at the bonds indicated by a [symbol]
X₁ and X₂ are each independently N, C, O, or S;
R₈ is H, halo, —OH, C₁₋₆ alkyl, C₂₋₆ alkenyl, C₂₋₆ alkynyl, —OH, —NRₐR_{b}, —(CH₂)ₙ—O—R_{c}, —O—(C₁₋₆ alkyl), —S(O)ₚRₑ, or —C(O)—R_{d};
R₉ is H, C₁₋₆ alkyl, substituted C₁₋₆ alkyl, heterocyclyl, substituted heterocyclyl or R₉ₐ, wherein R₉ₐ is:

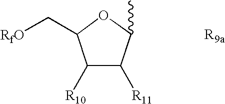

the binding being achieved at the bond indicated by a [symbol]
R₁₀ and R₁₁ are each independently H, halo, C₁₋₆ alkoxy, substituted C₁₋₆ alkoxy, —NRₐR_{b}, or —OH;
each Rₐ and R_{b} is independently H, C₁₋₆ alkyl, substituted C₁₋₆ alkyl, —C(O)R_{d}, C₆₋₁₀ aryl;
each R_{c} is independently H, phosphate, diphosphate, triphosphate, C₁₋₆ alkyl, or substituted C₁₋₆ alkyl;
each R_{d} is independently H, halo, C₁₋₆ alkyl, substituted C₁₋₆ alkyl, C₁₋₆ alkoxy, substituted C₁₋₆ alkoxy, —NH₂, —NH(C₁₋₆ alkyl), —NH(substituted C₁₋₆ alkyl), —N(C₁₋₆ alkyl)₂, —N(substituted C₁₋₆ alkyl)₂, C₆₋₁₀ aryl, or heterocyclyl;
each Rₑ is independently H, C₁₋₆ alkyl, substituted C₁₋₆ alkyl, C₆₋₁₀ aryl, substituted C₆₋₁₀ aryl, heterocyclyl, or substituted heterocyclyl;
each R_{f} is independently H, C₁₋₆ alkyl, substituted C-6 alkyl, —C(O)R_{d}, phosphate, diphosphate, or triphosphate;
each n is independently 0, 1, 2, or 3;
each p is independently 0, 1, or 2; or
or (g) a pharmaceutically acceptable salt of any of (a) to (f), a tautomer of any of (a) to (f), or a pharmaceutically acceptable salt of the tautomer.
Loxoribine (7-allyl-8-oxoguanosine) [77].
Compounds disclosed in reference 78, including: Acylpiperazine compounds, Indoledione compounds, Tetrahydraisoquinoline (THIQ) compounds, Benzocyclodione compounds, Aminoazavinyl compounds, Aminobenzimidazole quinolinone (ABIQ) compounds [79,80], Hydrapthalamide compounds, Benzophenone compounds, Isoxazole compounds, Sterol compounds, Quinazilinone compounds, Pyrrole compounds [81], Anthraquinone compounds, Quinoxaline compounds, Triazine compounds, Pyrazalopyrimidine compounds, and Benzazole compounds [82].
Compounds disclosed in reference 83, including 3,4-di(1H-indol-3-yl)-1H-pyrrole-2,5-diones, staurosporine analogs, derivatized pyridazines, chromen-4-ones, indolinones, quinazolines, and nucleoside analogs.
An aminoalkyl glucosaminide phosphate derivative, such as RC-529 [84,85].
A phosphazene, such as poly[di(carboxylatophenoxy)phosphazene] (“PCPP”) as described, for example, in references 86 and 87.
Small molecule immunopotentiators (SMIPs) such as:
N2-methyl-1-(2-methylpropyl)-1H-imidazo[4,5-c]quinoline-2,4-diamine
N2,N2-dimethyl-1-(2-methylpropyl)-1H-imidazo[4,5-c]quinoline-2,4-diamine
N2-ethyl-N2-methyl-1-(2-methylpropyl)-1H-imidazo[4,5-c]quinoline-2,4-diamine
N2-methyl-1-(2-methylpropyl)-N2-propyl-1H-imidazo[4,5-c]quinoline-2,4-diamine
1-(2-methylpropyl)-N2-propyl-1H-imidazo[4,5-c]quinoline-2,4-diamine
N2-butyl-1-(2-methylpropyl)-1H-imidazo[4,5-c]quinoline-2,4-diamine
N2-butyl-N2-methyl-1-(2-methylpropyl)-1H-imidazo[4,5-c]quinoline-2,4-diamine
N2-methyl-1-(2-methylpropyl)-N2-pentyl-1H-imidazo[4,5-c]quinoline-2,4-diamine
N2-methyl-1-(2-methylpropyl)-N2-prop-2-enyl-1H-imidazo[4,5-c]quinoline-2,4-diamine
1-(2-methylpropyl)-2-[(phenylmethyl)thio]-1H-imidazo[4,5-c]quinolin-4-amine
1-(2-methylpropyl)-2-(propylthio)-1H-imidazo[4,5-c]quinolin-4-amine
2-[[4-amino-1-(2-methylpropyl)-1H-imidazo[4,5-c]quinolin-2-yl](methyl)amino]ethanol
2-[[4-amino-1-(2-methylpropyl)-1H-imidazo[4,5-c]quinolin-2-yl](methyl)amino]ethyl acetate
4-amino-1-(2-methylpropyl)-1,3-dihydro-2H-imidazo[4,5-c]quinolin-2-one
N2-butyl-1-(2-methylpropyl)-N4,N4-bis(phenylmethyl)-1H-imidazo[4,5-c]quinoline-2,4-diamine
N2-butyl-N2-methyl-1-(2-methylpropyl)-N4,N4-bis(phenylmethyl)-1H-imidazo[4,5-c]quinoline-2,4-diamine
N2-methyl-1-(2-methylpropyl)-N4,N4-bis(phenylmethyl)-1H-imidazo[4,5-c]quinoline-2,4-diamine
N2,N2-dimethyl-1-(2-methylpropyl)-N4,N4-bis(phenylmethyl)-1H-imidazo[4,5-c]quinoline-2,4-diamine
1-{4-amino-2-[methyl(propyl)amino]-1H-imidazo[4,5-c]quinolin-1-yl}-2-methylpropan-2-ol
1-[4-amino-2-(propylamino)-1H-imidazo[4,5-c]quinolin-1-yl]-2-methylpropan-2-ol
N4,N4-dibenzyl-1-(2-methoxy-2-methylpropyl)-N2-propyl-H-imidazo[4,5-c]quinoline-2,4-diamine.
   Saponins [chapter 22 of ref. 118], which are a heterologous group of sterol glycosides and triterpenoid glycosides that are found in the bark, leaves, stems, roots and even flowers of a wide range of plant species. Saponin from the bark of the Quillaia saponaria Molina tree have been widely studied as adjuvants. Saponin can also be commercially obtained from Smilax ornata (sarsaprilla), Gypsophilla paniculata (brides veil), and Saponaria officianalis (soap toot). Saponin adjuvant formulations include purified formulations, such as QS21, as well as lipid formulations, such as ISCOMs. QS21 is marketed as Stimulon™. Saponin compositions have been purified using HPLC and RP-HPLC. Specific purified fractions using these techniques have been identified, including QS7, QS17, QS18, QS21, QH-A, QH-B and QH-C. Preferably, the saponin is QS21. A method of production of QS21 is disclosed in ref. 88. Saponin formulations may also comprise a sterol, such as cholesterol [89]. Combinations of saponins and cholesterols can be used to form unique particles called immunostimulating complexs (ISCOMs) [chapter 23 of ref. 118]. ISCOMs typically also include a phospholipid such as phosphatidylethanolamine or phosphatidylcholine. Any known saponin can be used in ISCOMs. Preferably, the ISCOM includes one or more of QuilA, QHA & QHC. ISCOMs are further described in refs. 89-91. Optionally, the ISCOMS may be devoid of additional detergent [92]. A review of the development of saponin based adjuvants can be found in refs. 93 & 94.
   Bacterial ADP-ribosylating toxins (e.g. the E. coli heat labile enterotoxin “LT”, cholera toxin “CT”, or pertussis toxin “PT”) and detoxified derivatives thereof, such as the mutant toxins known as LT-K63 and LT-R_{72 [95}]. The use of detoxified ADP-ribosylating toxins as mucosal adjuvants is described in ref. 96 and as parenteral adjuvants in ref. 97.
   Bioadhesives and mucoadhesives, such as esterified hyaluronic acid microspheres [98] or chitosan and its derivatives [99].
   Microparticles (i.e. a particle of ˜100 nm to ˜150 μm in diameter, more preferably ˜200 nm to ˜30 μm in diameter, or ˜500 nm to ˜10 μm in diameter) formed from materials that are biodegradable and non-toxic (e.g. a poly(α-hydroxy acid), a polyhydroxybutyric acid, a polyorthoester, a polyanhydride, a polycaprolactone, etc.), with poly(lactide-co-glycolide) being preferred, optionally treated to have a negatively-charged surface (e.g. with SDS) or a positively-charged surface (e.g. with a cationic detergent, such as CTAB).
   Liposomes (Chapters 13 & 14 of ref. 118). Examples of liposome formulations suitable for use as adjuvants are described in refs. 100-102.
   Polyoxyethylene ethers and polyoxyethylene esters [103]. Such formulations further include polyoxyethylene sorbitan ester surfactants in combination with an octoxynol [104] as well as polyoxyethylene alkyl ethers or ester surfactants in combination with at least one additional non-ionic surfactant such as an octoxynol [105]. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauryl ether.
   Muramyl peptides, such as N-acetylmuramyl-L-threonyl-D-isoglutamine (“thr-MDP”), N-acetyl-normuramyl-L-alanyl-D-isoglutamine (nor-MDP), N-acetylglucsaminyl-N-acetylmuramyl-L-Al-D-isoglu-L-Ala-dipalmitoxy propylamide (“DTP-DPP”, or “Theramide™), N-acetylmuramyl-L-alanyl-D-isoglutaminyl-L-alanine-2-(1′-2′dipalmitoyl-sn-glycero-3-hydroxyphosphoryloxy)-ethylamine (“MTP-PE”).
   An outer membrane protein proteosome preparation prepared from a first Gram-negative bacterium in combination with a liposaccharide (LPS) preparation derived from a second Gram-negative bacterium, wherein the outer membrane protein proteosome and LPS preparations form a stable non-covalent adjuvant complex. Such complexes include “IVX-908”, a complex comprised of Neisseria meningitidis outer membrane and LPS.
   Methyl inosine 5′-monophosphate (“MIMP”) [106].
   A polyhydroxlated pyrrolizidine compound [107], such as one having formula:

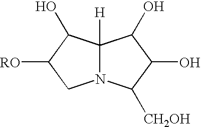

where R is selected from the group comprising hydrogen, straight or branched, unsubstituted or substituted, saturated or unsaturated acyl, alkyl (e.g. cycloalkyl), alkenyl, alkynyl and aryl groups, or a pharmaceutically acceptable salt or derivative thereof. Examples include, but are not limited to: casuarine, casuarine-6-α-D-glucopyranose, 3-epi-casuarine, 7-epi-casuarine, 3,7-diepi-casuarine, etc.
A gamma inulin [108] or derivative thereof, such as algammulin.
A compound of formula I, II or III, or a salt thereof:

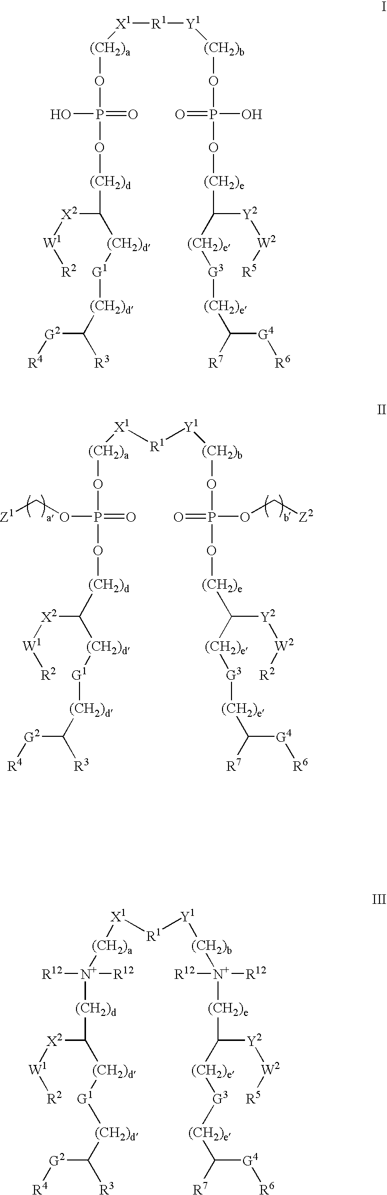

as defined in reference 109, such as ‘ER 803058’, ‘ER 803732’, ‘ER 804053’, ER 804058’, ‘ER 804059’, ‘ER 804442’, ‘ER 804680’, ‘ER 804764’, ER 803022 or ‘ER 804057’ e.g.:

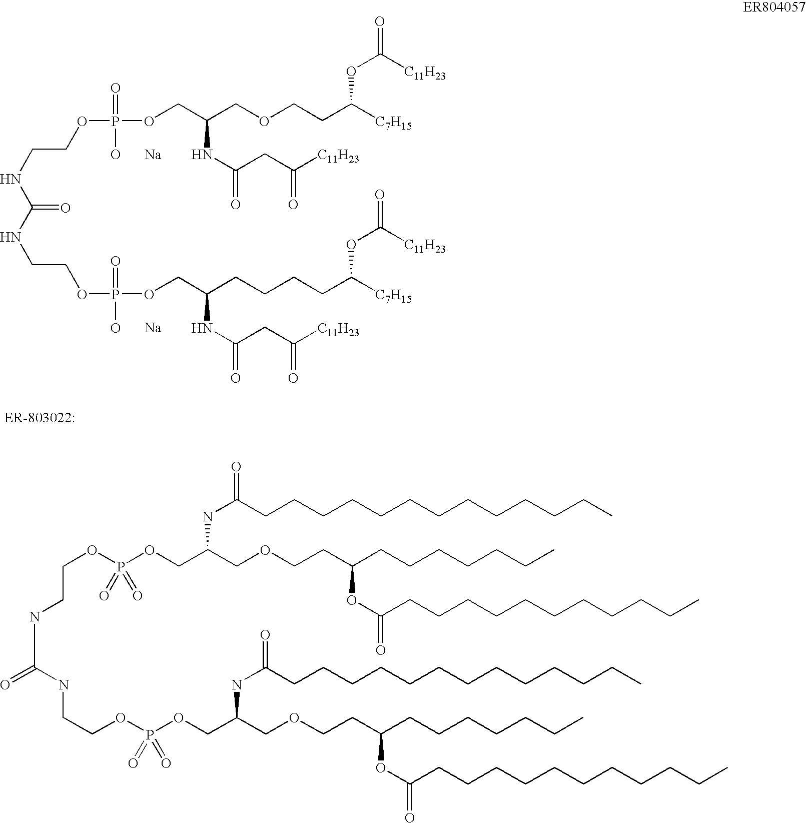

Derivatives of lipid A from Escherichia coli such as OM-174 (described in refs. 110 & 111).
A formulation of a cationic lipid and a (usually neutral) co-lipid, such as aminopropyl-dimethyl-myristoleyloxy-propanaminium bromide-diphytanoylphosphatidyl-ethanolamine (“Vaxfectin™”) or aminopropyl-dimethyl-bis-dodecyloxy-propanaminium bromide-dioleoylphosphatidyl-ethanolamine (“GAP-DLRIE:DOPE”). Formulations containing (±)—N-(3-aminopropyl)-N,N-dimethyl-2,3-bis(syn-9-tetradeceneyloxy)-1-propanaminium salts are preferred [112].
Compounds containing lipids linked to a phosphate-containing acyclic backbone, such as the TLR₄ antagonist E5564 [113,114]:

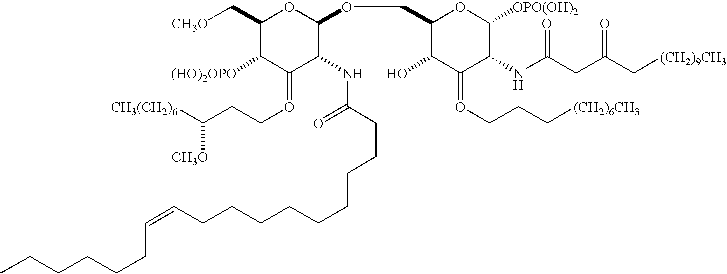

These and other adjuvant-active substances are discussed in more detail in references 118 & 119.

Compositions may include two or more of said adjuvants.

Antigens and adjuvants in a composition will typically be in admixture.

### Oil-in-Water Emulsion Adjuvants

Oil-in-water emulsions are particularly useful as adjuvants. Various such emulsions are known, and they typically include at least one oil and at least one surfactant, with the oil(s) and surfactant(s) being biodegradable (metabolisable) and biocompatible. The oil droplets in the emulsion are generally less than 5 μm in diameter, and may even have a sub-micron diameter, with these small sizes being achieved with a microfluidiser to provide stable emulsions. Droplets with a size less than 220 nm are preferred as they can be subjected to filter sterilization.

The invention can be used with oils such as those from an animal (such as fish) or vegetable source. Sources for vegetable oils include nuts, seeds and grains. Peanut oil, soybean oil, coconut oil, and olive oil, the most commonly available, exemplify the nut oils. Jojoba oil can be used e.g. obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. Most fish contain metabolizable oils which may be readily recovered. For example, cod liver oil, shark liver oils, and whale oil such as spermaceti exemplify several of the fish oils which may be used herein. A number of branched chain oils are synthesized biochemically in 5-carbon isoprene units and are generally referred to as terpenoids. Shark liver oil contains a branched, unsaturated terpenoids known as squalene, 2,6,10,15,19,23-hexamethyl-2,6,10,14,18,22-tetracosahexaene, which is particularly preferred herein. Squalane, the saturated analog to squalene, is also a preferred oil. Fish oils, including squalene and squalane, are readily available from commercial sources or may be obtained by methods known in the art. Other preferred oils are the tocopherols (see below). Mixtures of oils can be used.

Surfactants can be classified by their ‘HLB’ (hydrophile/lipophile balance). Preferred surfactants of the invention have a HLB of at least 10, preferably at least 15, and more preferably at least 16. The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); and sorbitan esters (commonly known as the SPANs), such as sorbitan trioleate (Span 85) and sorbitan monolaurate. Preferred surfactants for including in the emulsion are Tween 80 (polyoxyethylene sorbitan monooleate), Span 85 (sorbitan trioleate), lecithin and Triton X-100. Mixtures of surfactants can be used e.g. Tween 80/Span 85 mixtures.

Specific oil-in-water emulsion adjuvants useful with the invention include, but are not limited to:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as ‘MF59’ [115-117], as described in more detail in Chapter 10 of ref. 118 and chapter 12 of ref. 119. The MF59 emulsion advantageously includes citrate ions e.g. 110 mM sodium citrate buffer.
An emulsion of squalene, a tocopherol, and Tween 80. The emulsion may include phosphate buffered saline. It may also include Span 85 (e.g. at 1%) and/or lecithin. These emulsions may have from 2 to 10% squalene, from 2 to 10% tocopherol and from 0.3 to 3% Tween 80, and the weight ratio of squalene:tocopherol is preferably ≦1 as this provides a more stable emulsion. One such emulsion can be made by dissolving Tween 80 in PBS to give a 2% solution, then mixing 90 ml of this solution with a mixture of (5 g of DL-α-tocopherol and 5 ml squalene), then microfluidising the mixture. The resulting emulsion may have submicron oil droplets e.g. with an average diameter of between 100 and 250 nm, preferably about 180 nm.
An emulsion of squalene, a tocopherol, and a Triton detergent (e.g. Triton X-100).
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant [120] (0.05-1% Thr-MDP, 5% squalane, 2.5% Pluronic L121 and 0.2% polysorbate 80). It can also be used without the Thr-MDP, as in the “AF” adjuvant [121] (5% squalane, 1.25% Pluronic L121 and 0.2% polysorbate 80). Microfluidisation is preferred.
An emulsion having from 0.5-50% of an oil, 0.1-10% of a phospholipid, and 0.05-5% of a non-ionic surfactant. As described in reference 122, preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolisable oil (such as light mineral oil) and at least one surfactant (such as lecithin, Tween 80 or Span 80). Additives may be included, such as QuilA saponin, cholesterol, a saponin-lipophile conjugate (such as GPI-0100, described in reference 123, produced by addition of aliphatic amine to desacylsaponin via the carboxyl group of glucuronic acid), dimethyldioctadecylammonium bromide and/or N,N-dioctadecyl-N,N-bis(2-hydroxyethyl)propanediamine.
An emulsion in which a saponin (e.g. QuilA or QS21) and a sterol (e.g. a cholesterol) are associated as helical micelles [124].

The emulsions may be mixed with antigen extemporaneously, at the time of delivery. Thus the adjuvant and antigen may be kept separately in a packaged or distributed vaccine, ready for final formulation at the time of use. The antigen will generally be in an aqueous form, such that the vaccine is finally prepared by mixing two liquids. The volume ratio of the two liquids for mixing can vary (e.g. between 5:1 and 1:5) but is generally about 1:1.

### Aluminum Salt Adjuvants

The adjuvants known as aluminum hydroxide and aluminum phosphate may be used. These names are conventional, but are used for convenience only, as neither is a precise description of the actual chemical compound which is present (e.g. see chapter 9 of reference 118). The invention can use any of the “hydroxide” or “phosphate” adjuvants that are in general use as adjuvants.

The adjuvants known as “aluminium hydroxide” are typically aluminium oxyhydroxide salts, which are usually at least partially crystalline. Aluminium oxyhydroxide, which can be represented by the formula AlO(OH), can be distinguished from other aluminium compounds, such as aluminium hydroxide Al(OH)₃, by infrared (IR) spectroscopy, in particular by the presence of an adsorption band at 1070 cm⁻¹ and a strong shoulder at 3090-33100 cm⁻¹ [chapter 9 of ref. 118]. The degree of crystallinity of an aluminium hydroxide adjuvant is reflected by the width of the diffraction band at half height (WHH), with poorly-crystalline particles showing greater line broadening due to smaller crystallite sizes. The surface area increases as WHH increases, and adjuvants with higher WHH values have been seen to have greater capacity for antigen adsorption. A fibrous morphology (e.g. as seen in transmission electron micrographs) is typical for aluminium hydroxide adjuvants. The pI of aluminium hydroxide adjuvants is typically about 11 i.e. the adjuvant itself has a positive surface charge at physiological pH. Adsorptive capacities of between 1.8-2.6 mg protein per mg Al⁺⁺⁺ at pH 7.4 have been reported for aluminium hydroxide adjuvants.

The adjuvants known as “aluminium phosphate” are typically aluminium hydroxyphosphates, often also containing a small amount of sulfate (i.e. aluminium hydroxyphosphate sulfate). They may be obtained by precipitation, and the reaction conditions and concentrations during precipitation influence the degree of substitution of phosphate for hydroxyl in the salt. Hydroxyphosphates generally have a PO₄/Al molar ratio between 0.3 and 1.2. Hydroxyphosphates can be distinguished from strict AlPO₄ by the presence of hydroxyl groups. For example, an IR spectrum band at 3164 cm⁻¹ (e.g. when heated to 200° C.) indicates the presence of structural hydroxyls [ch.9 of ref. 118].

The PO₄/Al³⁺ molar ratio of an aluminium phosphate adjuvant will generally be between 0.3 and 1.2, preferably between 0.8 and 1.2, and more preferably 0.95±0.1. The aluminium phosphate will generally be amorphous, particularly for hydroxyphosphate salts. A typical adjuvant is amorphous aluminium hydroxyphosphate with PO₄/Al molar ratio between 0.84 and 0.92, included at 0.6 mg Al³⁺/ml. The aluminium phosphate will generally be particulate (e.g. plate-like morphology as seen in transmission electron micrographs). Typical diameters of the particles are in the range 0.5-20 μm (e.g. about 5-10 μm) after any antigen adsorption. Adsorptive capacities of between 0.7-1.5 mg protein per mg Al⁺⁺⁺ at pH 7.4 have been reported for aluminium phosphate adjuvants.

The point of zero charge (PZC) of aluminium phosphate is inversely related to the degree of substitution of phosphate for hydroxyl, and this degree of substitution can vary depending on reaction conditions and concentration of reactants used for preparing the salt by precipitation. PZC is also altered by changing the concentration of free phosphate ions in solution (more phosphate=more acidic PZC) or by adding a buffer such as a histidine buffer (makes PZC more basic). Aluminium phosphates used according to the invention will generally have a PZC of between 4.0 and 7.0, more preferably between 5.0 and 6.5 e.g. about 5.7.

Suspensions of aluminium salts used to prepare compositions of the invention may contain a buffer (e.g. a phosphate or a histidine or a Tris buffer), but this is not always necessary. The suspensions are preferably sterile and pyrogen-free. A suspension may include free aqueous phosphate ions e.g. present at a concentration between 1.0 and 20 mM, preferably between 5 and 15 mM, and more preferably about 10 mM. The suspensions may also comprise sodium chloride.

The invention can use a mixture of both an aluminium hydroxide and an aluminium phosphate. In this case there may be more aluminium phosphate than hydroxide e.g. a weight ratio of at least 2:1 e.g. ≧5:1, ≧6:1, ≧7:1, ≧8:1, ≧9:1, etc.

The concentration of Al⁺⁺⁺ in a composition for administration to a patient is preferably less than 10 mg/ml e.g. ≦5 mg/ml, ≦4 mg/ml, ≦3 mg/ml, ≦2 mg/ml, ≦1 mg/ml, etc. A preferred range is between 0.3 and 1 mg/ml.

### Kits of the Invention

Where a composition includes two components for delivery to a patient, such as a Env/Tat complex and an adjuvant, these may be mixed during manufacture, or they may be mixed extemporaneously, at the time of delivery. Thus the invention provides kits including the various components ready for mixing. The kit allows the adjuvant and the complex to be kept separately until the time of use. This arrangement is particularly useful when using an oil-in-water emulsion adjuvant.

The components are physically separate from each other within the kit, and this separation can be achieved in various ways. For instance, the two components may be in two separate containers, such as vials. The contents of the two vials can then be mixed e.g. by removing the contents of one vial and adding them to the other vial, or by separately removing the contents of both vials and mixing them in a third container.

In a preferred arrangement, one of the kit components is in a syringe and the other is in a container such as a vial. The syringe can be used (e.g. with a needle) to insert its contents into the second container for mixing, and the mixture can then be withdrawn into the syringe. The mixed contents of the syringe can then be administered to a patient, typically through a new sterile needle. Packing one component in a syringe eliminates the need for using a separate syringe for patient administration.

In another preferred arrangement, the two kit components are held together but separately in the same syringe e.g. a dual-chamber syringe, such as those disclosed in references 125-132 etc. When the syringe is actuated (e.g. during administration to a patient) then the contents of the two chambers are mixed. This arrangement avoids the need for a separate mixing step at the time of use.

The kit components will generally be in aqueous form. In some arrangements, a component (typically the antigen component rather than the adjuvant component) is in dry form (e.g. in a lyophilised form), with the other component being in aqueous form. The two components can be mixed in order to reactivate the dry component and give an aqueous composition for administration to a patient. A lyophilised component will typically be located within a vial rather than a syringe. Dried components may include stabilizers such as lactose, sucrose or mannitol, as well as mixtures thereof e.g. lactose/sucrose mixtures, sucrose/mannitol mixtures, etc. One possible arrangement uses an aqueous adjuvant component in a pre-filled syringe and a lyophilised antigen component in a vial.

### Methods of Treatment, and Administration of Vaccines

The invention provides a method of raising an immune response in a patient, comprising the step of administering a composition of the invention to the patient. The compositions of the invention are particularly suitable for administration to human patients, but can also be administered to other mammals for investigational purposes, for raising antisera, etc.

The invention also provides a kit or composition of the invention for use as a medicament.

The invention also provides the use of an Env/Tat complex of the invention in the manufacture of a medicament for raising an immune response in a patient.

Compositions of the invention can be administered in various ways. The most preferred immunisation route is by injection (e.g. intramuscular, subcutaneous, intravenous), but other available routes include, but are not limited to, intranasal, oral, intradermal, transcutaneous, transdermal, pulmonary, etc.

Treatment can be by a single dose schedule or a multiple dose schedule. Multiple doses may be used in a primary immunisation schedule and/or in a booster immunisation schedule. In a multiple dose schedule the various doses may be given by the same or different routes e.g. a parenteral prime and mucosal boost, a mucosal prime and parenteral boost, etc. Administration of more than one dose (typically two doses) is typical. Multiple doses will typically be administered at least 1 week apart (e.g. about 2 weeks, about 3 weeks, about 4 weeks, about 6 weeks, about 8 weeks, etc.).

### General

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x means, for example, x±10%.

Unless specifically stated, a process comprising a step of mixing two or more components does not require any specific order of mixing. Thus components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encaphalopathies (TSEs), and in particular free from bovine spongiform encephalopathy (BSE). Overall, it is preferred to culture cells in the total absence of animal-derived materials.

Where a protein or a complex “binds specifically” to a particular target (e.g. to CD4 or to a monoclonal antibody), it will typically bind to that target with at least 10-fold greater affinity than to a control protein e.g. than to CD3 or than to an anti-Rev antibody. Specific binding and non-specific binding can be distinguished by standard techniques e.g. by checking the effect of control proteins on the interaction, by checking dose-responsiveness, etc.

The term “polypeptide” refers to amino acid polymers of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art. Polypeptides can occur as single chains or associated chains. Polypeptides of the invention can be naturally or non-naturally glycosylated (i.e. the polypeptide has a glycosylation pattern that differs from the glycosylation pattern found in the corresponding naturally occurring polypeptide).

Env and Tat polypeptides for use with the invention can be prepared in many ways e.g. by chemical synthesis (in whole or in part), by digesting longer polypeptides using proteases, by translation from RNA, by purification from cell culture (e.g. from recombinant expression), from the organism itself (e.g. after bacterial culture, or direct from patients), etc. A preferred method for production of peptides <40 amino acids long involves in vitro chemical synthesis [133,134]. Solid-phase peptide synthesis is particularly preferred, such as methods based on tBoc or Fmoc [135] chemistry. Enzymatic synthesis [136] may also be used in part or in full. As an alternative to chemical synthesis, biological synthesis may be used e.g. the polypeptides may be produced by translation. This may be carried out in vitro or in vivo. Biological methods are in general restricted to the production of polypeptides based on L-amino acids, but manipulation of translation machinery (e.g. of aminoacyl tRNA molecules) can be used to allow the introduction of D-amino acids (or of other non natural amino acids, such as iodotyrosine or methylphenylalanine, azidohomoalanine, etc.) [137]. Where D-amino acids are included, however, it is preferred to use chemical synthesis. Polypeptides of the invention may have covalent modifications at the C-terminus and/or N-terminus.

Env and Tat polypeptides can take various forms (e.g. native, fusions, glycosylated, non-glycosylated, lipidated, non-lipidated, phosphorylated, non-phosphorylated, myristoylated, non-myristoylated, monomeric, multimeric, particulate, denatured, etc.). For Env, oligomeric glycosylated polypeptides are preferred. Monomeric polypeptides are preferred.

Env and Tat polypeptides are preferably provided in purified or substantially purified form i.e. substantially free from other polypeptides (e.g. free from naturally-occurring polypeptides), particularly from other HIV or host cell polypeptides, and are generally at least about 50% pure (by weight), and usually at least about 90% pure i.e. less than about 50%, and more preferably less than about 10% (e.g. 5% or less) of a composition is made up of other expressed polypeptides.

### EXAMPLES

### Non-Covalent Binding of Env and Tat

Four forms of Env protein were prepared from the SF162 strain of HIV-1: gp120; gp120ΔV2; gp140; and gp140ΔV2. The gp120 molecules are monomeric whereas the gp140 molecules are trimeric. These four proteins have previously been described (e.g. refs. 12, 138 & 139). Briefly, the sequences encoding the Env ectodomain from HIV-1 SF162 and HIV-1 SF162ΔV2 isolates were codon modified as described previously [138], and constructed synthetically as a 2.1-kb EcoRI-XbaI DNA fragment. The gene cassettes contained the protein-encoding region of the Env proteins fused in frame to the human tissue plasminogen activator (TPA) signal sequence for efficient secretion. In order to stabilize the oligomeric structure of the encoded oligomeric proteins, the primary (REKR) and secondary (KAKRR) protease cleavage sites in the Env polypeptides were modified [138]. The resulting Env expression cassettes (gp120SF162, gp120SF162ΔV2, gp140SF162 and gp140SF162ΔV2) were cloned into the EcoRI-XbaI sites of the pCMV3 expression vector for transient transfection of 293 cells and also for the development of stable CHO cell lines. This vector contains the cytomegalovirus enhancer/promoter elements, an ampicillin resistance gene, and sequences encoding a fusion protein composed of dihydrofolate reductase and an attenuated neomycin resistance protein.

Stable CHO cell lines secreting gp120SF162, gp120ΔV2SF162, gp140SF162, and gp140SF162ΔV2 were derived by using DG-44 cells with a double deletion in the dihydrofolate reductase gene, thus making the cell line dependent on the addition of hypoxanthine, glycine, and thymidine to the growth medium, following the experimental protocol described previously [138,139].

CHO cell clones producing the protein of interest were used to seed a 3-liter bioreactor for each protein. Bioreactors were monitored daily for cell density, pH, CO₂, and O₂ concentration, etc. The structure, conformation, and expression levels of secreted Env were monitored weekly.

Materials from the best producer clone was concentrated 20-fold through a 100-kDa-pore-size membrane filter and stored at −80° C. in presence of 1 mM EDTA and 1 mM EGTA.

All the envelope proteins were purified following the strategy described previously [139]. Briefly, the concentrated CHO cell supernatant was loaded onto a Galanthus Nivalis-agarose column (GNA) equilibrated with 20 mM Tris-100 mM NaCl (pH 7.4). Bound Env was eluted with 500 mM methyl mannose pyranoside. The eluate after the GNA column was loaded onto a DEAE column equilibrated with a buffer containing 20 mM Tris, 100 mM NaCl (pH 8.0). Under these conditions, Env does not bind to the column, but contaminating proteins are retained on the column. The DEAE flow through was adjusted to 10 mM PO₄ concentration, pH was adjusted to 6.8, and the flow through was loaded onto a ceramic hydroxyapatite (CHAP) column equilibrated with buffer containing 10 mM Na₂HPO₄, 100 mM NaCl (pH 6.8). Under these conditions, the env proteins did not bind to CHAP column and were recovered in the flow through. During the purification process, fractions were analyzed by polyacrylamide gel electrophoresis (PAGE) both under reducing and denaturing and under native conditions following standard methods and also in a CD4 receptor-binding assay. Gels were stained with Coomassie brilliant blue or processed for immunoblotting. All the fractions containing Env monomer with and without V2 loop were pooled, concentrated, and stored frozen at −80° C. Peak fractions containing o-gp140SF162 and o-gp140 SF162ΔV2 were pooled, concentrated and fractionated on a 16×90 mm Superdex-200 column equilibrated with 10 mM NaCitrate plus 300 mM NaCl to separate monomer from trimer. The fractions containing Env protein in trimeric conformation, were pooled, concentrated and kept frozen at −80° C. until used.

Tat protein from strain BH10 was also expressed and purified.

Far-Western analysis was used to study the interaction between these Env and Tat proteins. Briefly, known amounts of Tat and Env proteins were incubated for 2 hours at 4° C., to form complexes. 5 μl of a monoclonal anti-Tat antibody (4.3 mg/ml) was then added and the mixture was incubated overnight at 4° C. 50 μl of protein A was then added (Protein A Sepharose beads, 50% solution) and the mixture was incubated for a further 2 hours at 4° C. with agitation. The mixture was then washed 3 times and eluted into 4× sample buffer in a volume of 50 μl. The eluted proteins were then separated by SDS-PAGE and transferred onto nitrocellulose using semi-dry transfer. The resulting blots were incubated first with an anti-Env polyclonal rabbit antibody. The blots were washed and incubated with an anti-rabbit secondary antibody conjugated to alexa fluor 780. Blots were then read on an Odyssey infrared detector.

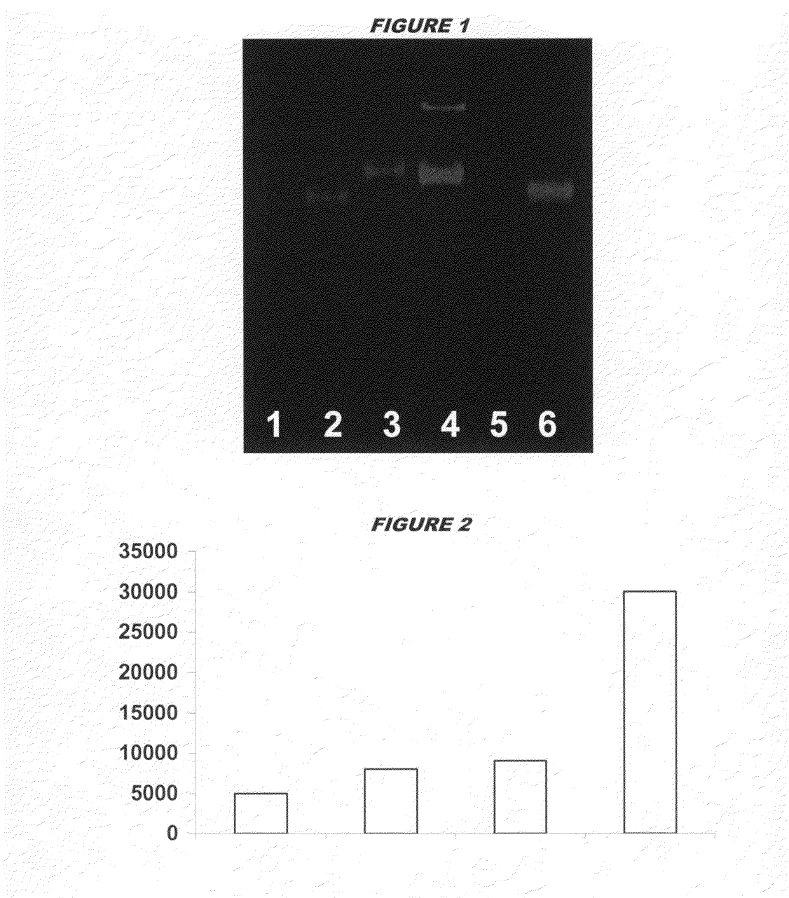

FIG. 1 shows the results of the Far-Western analysis using 1 μg Tat and 8 μg Env. Bands are clearly visible in lanes 2, 3, 4 and 6. FIG. 2 shows a quantitative analysis of the label intensity in lanes 1 to 4, which contain the Env/Tat mixtures. The lowest intensity was in lane 1 (gp120 monomer). Lanes 2 (gp120ΔV2 monomer) and 3 (gp140 trimer) showed similar intensities. The strongest intensity was seen in lane 4 (gp140ΔV2 trimer).

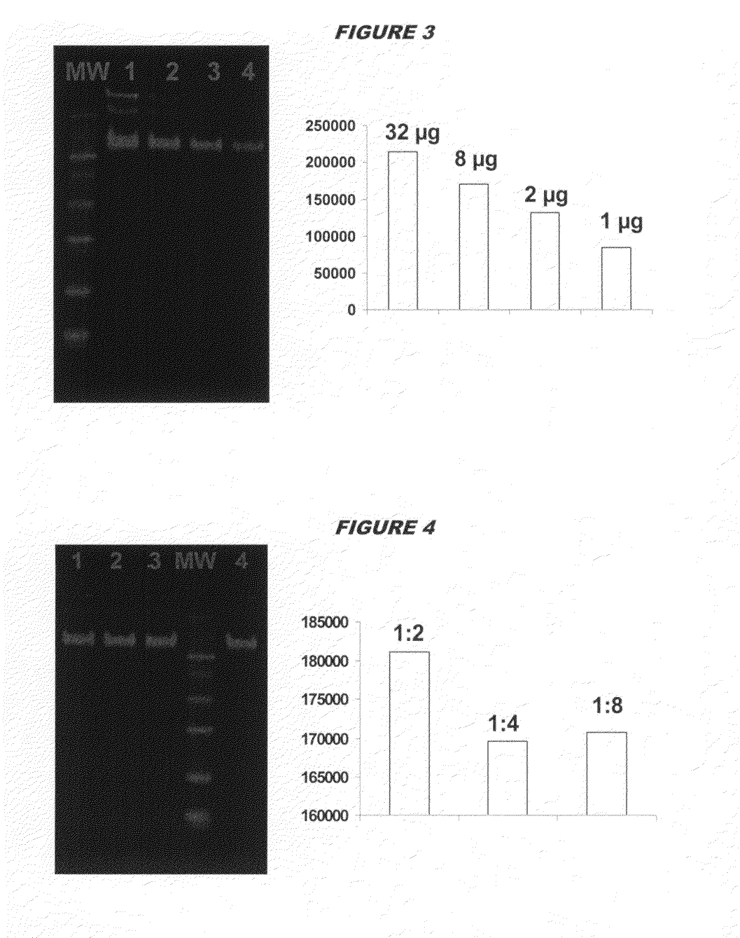

Further experiments using 1 μg Tat and varying amounts of Env (FIG. 3) confirmed that the interaction between Env and Tat is specific.

In the reverse experiment, where the amount of Env was fixed but varying amounts of Tat were used, different results were seen. The best interaction was observed when Env and Tat were mixed in the Env:Tat mass ratio of 1:2. Increased amounts of Tat had a detrimental effect on Env binding (FIG. 4).

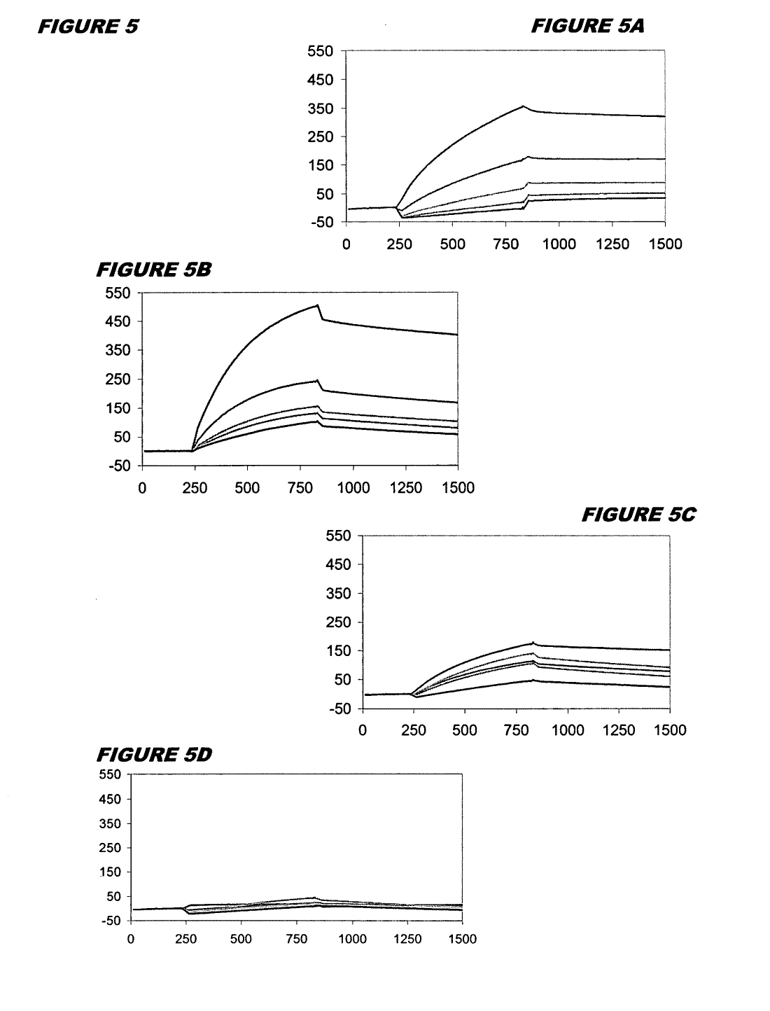

Surface plasmon resonance (SPR) was used to determine the strength of Env/Tat binding in a kinetic experiment. The results (FIG. 5) confirmed the results of the Far-Western assay. The dissociation constants for gp140ΔV2 trimer (FIG. 5A), the gp140 trimer (FIG. 5B), and gp120ΔV2 monomer (FIG. 5C) were 22 nM, 37 nM and 91 nM, respectively. The gp120 monomer did not bind to Tat at any concentration tested, and even under different experimental conditions.

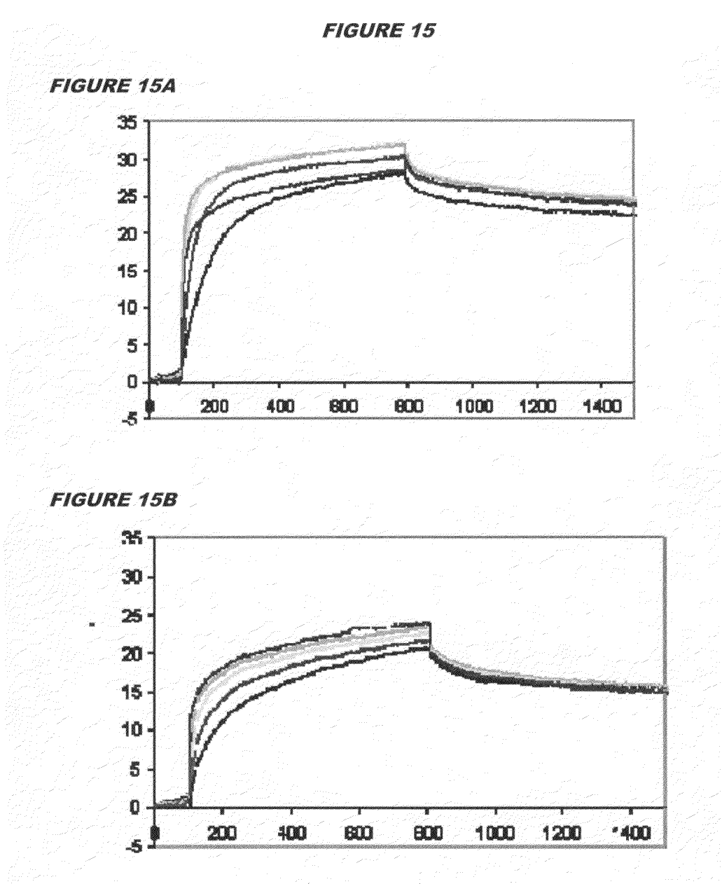

In further SPR experiments, Tat protein was immobilized on a CM4 chip and was exposed to Env protein from subtype C strain TV1. Different concentrations (63, 125, 250, and 1000 nM) of either native Env trimer (o-gp140 TV1) or ΔV2-Env trimer (o-gp140DV2 TV1) were tested. FIG. 15 shows the results.

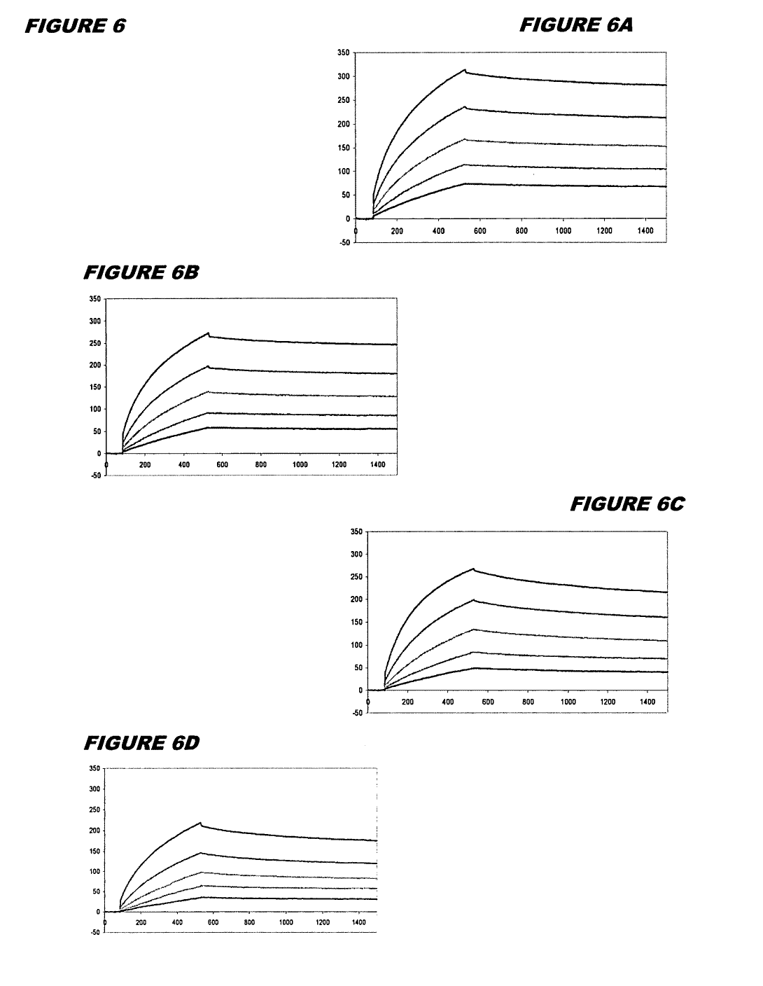

To determine if the lack of binding to gp120 by Tat was due to a functionally inert gp120, all of the Env proteins were analyzed for their ability to bind CD4 as a predictor of functional activity. All four Env proteins bound to CD4 with dissociation constants in the expected range (FIG. 6). Thus the monomeric gp120 was functional.

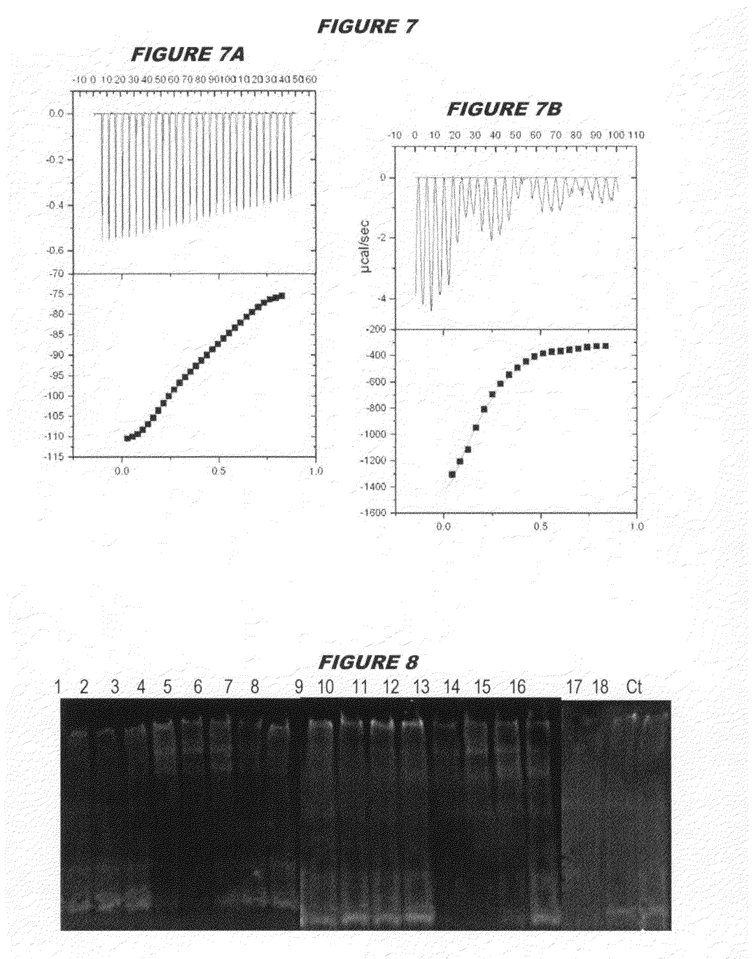

The interaction between Tat and Env trimers was also investigated using isothermal titration calorimetric analysis (ITC) in free solution. Preliminary ITC data were consistent with the previous experiments, showing that the gp140 trimer binds Tat more weakly than the gp140ΔV2 trimer (FIG. 7). The data also suggest that an Env trimer binds three Tat molecules e.g. each Env monomer has a single Tat binding site.

To investigate the site of Tat-binding on the Env protein, binding interactions with CD4 were compared. Tat did not compete for binding to CD4, and so the binding sites on Env for Tat and CD4 seem to be different.

### Covalent Linking of Env and Tat

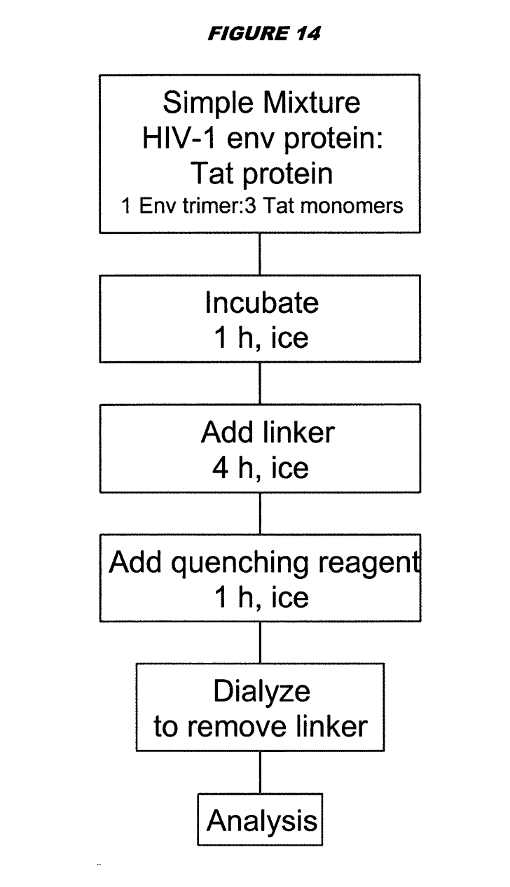

To stabilize the Env/Tat complexes, formaldehyde and glutaraldehyde were used as cross-linking reagents according to the reaction scheme illustrated in FIG. 14. They were tested under 18 different conditions:

| | | |
|---|---|---|
| 1: 0.06% | 7: 0.6% | 13: 0.02% |
| formaldehyde, | formaldehyde, | glutaraldehyde, |
| 24 hours | 2 hours | 8 hours |
| 2: 0.03% | 8: 0.3% | 14: 0.04% |
| formaldehyde, | formaldehyde, | glutaraldehyde, |
| 24 hours | 2 hours | 8 hours |
| 3: 0.12% | 9: 0.1% | 15: 0.01% |
| formaldehyde, | formaldehyde, | glutaraldehyde, |
| 24 hours | 2 hours | 8 hours |
| 4: 0.02% | 10: 0.06% | 16: 0.6% |
| glutaraldehyde, | formaldehyde, | formaldehyde, |
| 4 hours | 36 hours | 4 hours |
| 5: 0.04% | 11: 0.03% | 17: 0.3% |
| glutaraldehyde, | formaldehyde, | formaldehyde, |
| 4 hours | 36 hours | 4 hours |
| 6: 0.01% | 12: 0.12% | 18: 0.1% |
| glutaraldehyde, | formaldehyde, | formaldehyde, |
| 4 hours | 36 hours | 4 hours |

The resulting complexes were tested by various criteria, including: the presence of Env; the presence of Tat; the nature of crosslinking; the preservation of epitopes; and the preservation of binding activity. A control complex was also used with no cross-linking.

Env and Tat proteins were mixed as described above. Cross-linking reagents were added at various concentrations and reactions were allowed to proceed for various periods of time. Reactions were quenched and then dialysis was used to remove unreacted cross-linker reagents. The complexes were then analyzed by SDS-PAGE, Western blotting, Far-Western analysis, SPR and SEC-HPLC.

FIG. 8 shows Western blots using an anti-Tat antibody for labeling. Of the 18 reaction conditions, free Tat was absent in numbers 4-6 and 13-15 and instead migrated as molecular weight species. Thus glutaraldehyde cross-linking at between 0.01% and 0.04% for 4 to 8 hours is a prototypic set of conditions for effective covalent cross-linking.

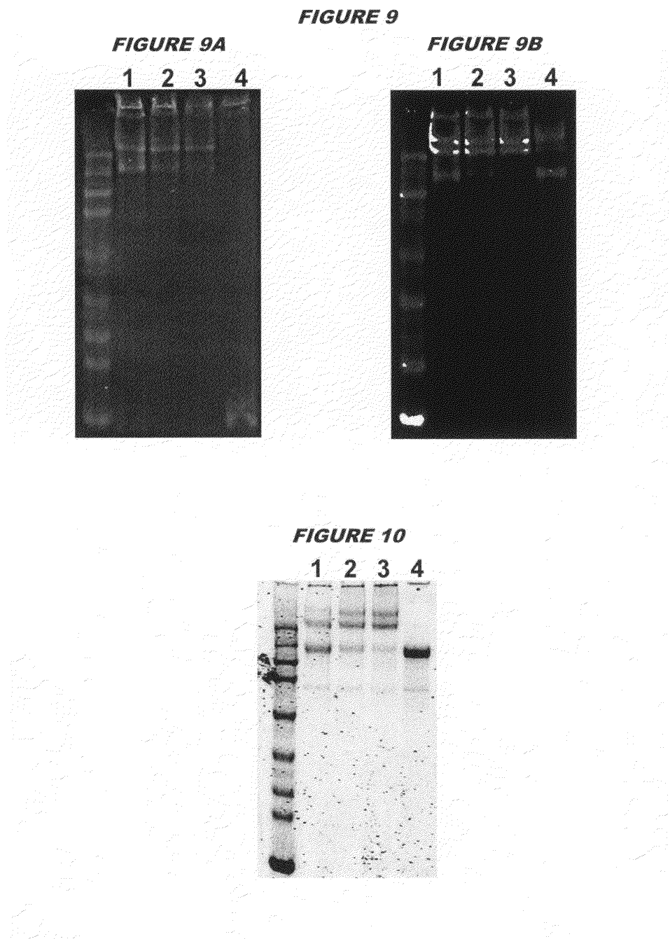

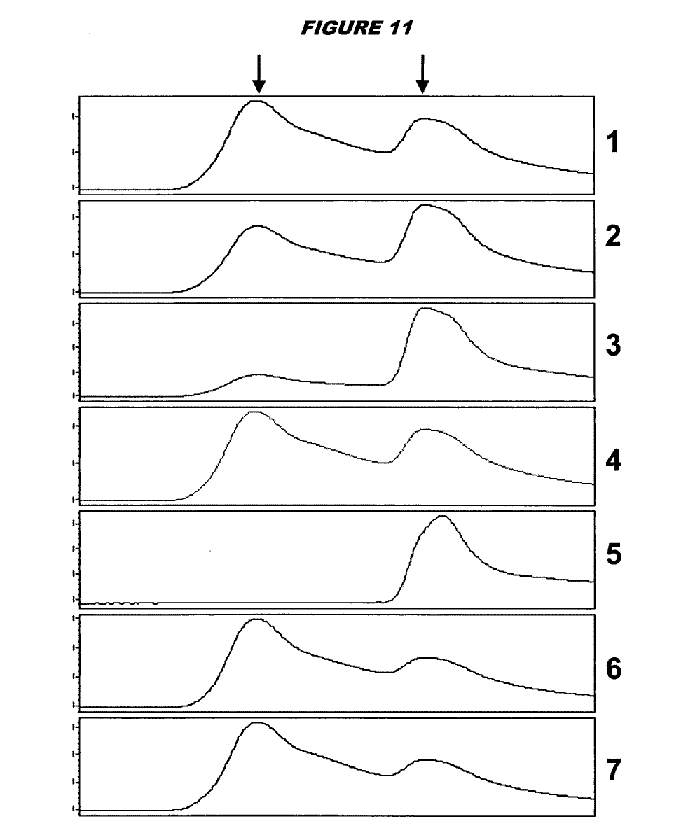

FIG. 9 shows Western blots using (9A) anti-Tat or (9B) anti-Env antibodies after using glutaraldehyde at 0.02%, 0.04% or 0.08%. These results confirmed that Env and Tat were both migrating as covalently-linked high MW complexes. SDS-PAGE analysis of the same complexes as in FIG. 9, under reducing and denaturing conditions, confirms a complex of >250 kDa, and the intensity of this species increases with the concentration of cross-linking reagent.

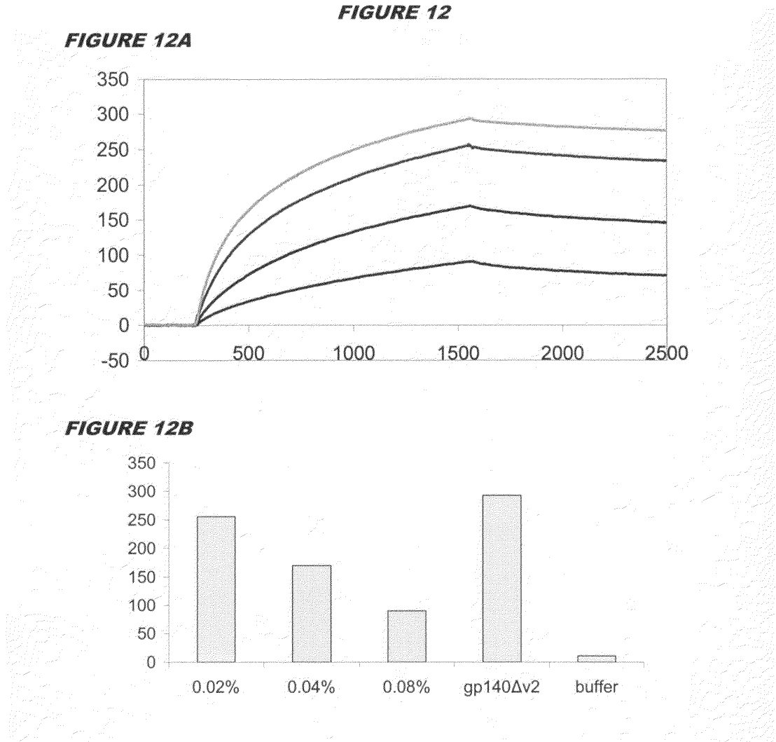

The effect of cross-linking on Env's CD4-binding activity was investigated. FIG. 11 shows the results of a SEC-HPLC analysis of the same complexes analyzed in FIG. 9. For comparison, an Env/Tat complex with no cross-linking, pure Env, pure Tat and a pre-cross-linking equimolar Env/Tat mixture (‘SM’) were also analyzed. The covalently-linked proteins retain the ability to bind to CD4 (compare lanes 1 and 4, and also 6). SPR was used for a similar analysis (FIG. 12). As the degree of cross-linking increases then CD4 binding decreases relative to gp140ΔV2 alone, but is still apparent even in the 0.08% sample and remains well above the level seen with the negative control.

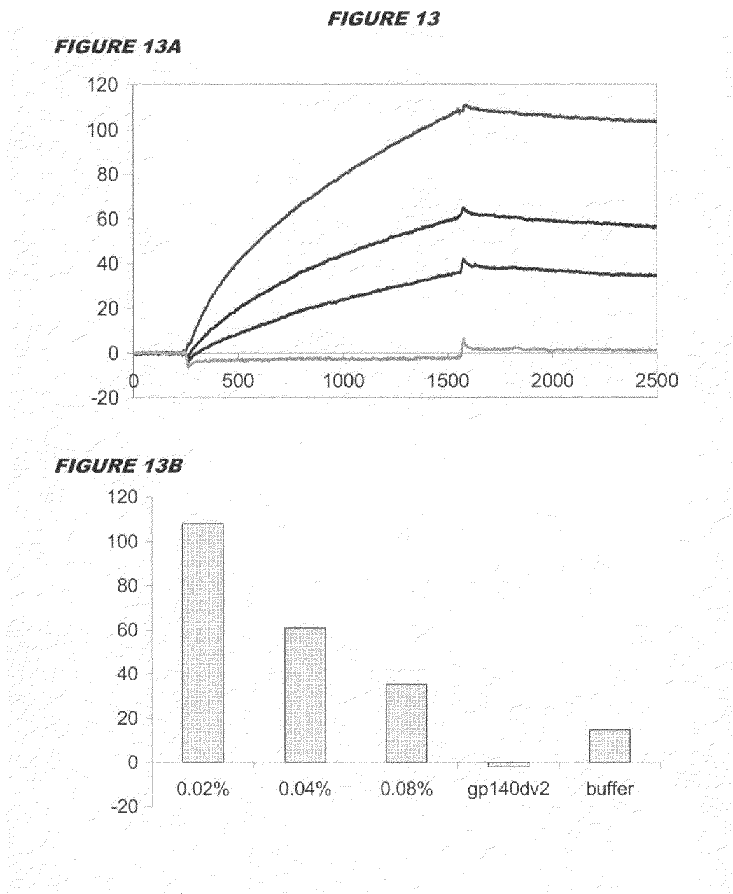

The effect of cross-linking on Tat epitopes was also investigated. FIG. 13 shows the results of SPR analysis. As for CD4 binding by Env, an increased level of cross-linking decreases the epitope's binding activity, but is still apparent even in the 0.08% sample and remains well above the control.

In combination, therefore, these results show that Env and Tat can be covalently cross-linked to form stable complexes, and that their binding activities can be maintained at functional levels.

It will be understood that the invention has been described by way of example only and modifications may be made whilst remaining within the scope and spirit of the invention.

### REFERENCES

### The Contents of which are Hereby Incorporated by Reference

[1] WO2005/090391.
[2] Ensoli et al. (2005) Microbes Infect 7:1392-9.
[3] Wong (1991) Chemistry of protein conjugation and cross-linking. ISBN 0-8493-5886-8.
[4] Hermanson (1996) Bioconjugate techniques. ISBN 0-12-342336-8.
[5] Robertson et al. (2000) Science 288:55-6.
[6] WO91/13906.
[7] http://www.aidsreagent.org/UploadDocs/ds4672_{—}003.pdf
[8] Rohr et al. (2003) J Virol. 77:5415-27.
[9] Avraham et al. (2004) J Immunol 173:6228-33.
[10] Liu et al. (2002) J Virol 76:6689-700.
[11] http://hiv-web.lanl.gov/
[12] WO00/39302.
[13] WO03/020876.
[14] WO2005/007808.
[15] WO03/004620.
[16] WO00/39304.
[17] U.S. Pat. No. 5,792,459.
[18] Zhang et al. (2001) J. Biol. Chem. 276:39577-85.
[19] Earl et al. (2001) J Virol 75:645-53.
[20] Barnett et al. (2001) J Virol 75:5526-40.
[21] Srivastava et al. (2003) J Virol 77:2310-20.
[22] WO91/15238.
[23] Geysen et al. (1984) PNAS USA 81:3998-4002.
[24] Carter (1994) Methods Mol Biol 36:207-223.
[25] Jameson, B A et al. 1988, CABIOS4(1):181-186.
[26] Raddrizzani & Hammer (2000) Brief Bioinform 1(2):179-189.
[27] De Lalla et al. (1999) J. Immunol. 163:1725-1729.
[28] Brusic et al. (1998) Bioinformatics 14(2):121-130
[29] Meister et al. (1995) Vaccine 13(6):581-591.
[30] Roberts et al. (1996) AIDS Res Hum Retroviruses 12(7):593-610.
[31] Maksyutov & Zagrebelnaya (1993) Comput Appl Biosci 9(3):291-297.
[32] Feller & de la Cruz (1991) Nature 349(6311):720-1.
[33] Hopp (1993) Peptide Research 6:183-190.
[34] Welling et al. (1985) FEBS Lett. 188:215-218.
[35] Davenport et al. (1995) Immunogenetics 42:392-297.
[36] Needleman & Wunsch (1970) J. Mol. Biol. 48:443-453.
[37] Rice et al. (2000) Trends Genet. 16:276-277.
[38] WO99/27958
[39] Gennaro (2000) Remington: The Science and Practice of Pharmacy. 20th edition, ISBN: 0683306472.
[40] U.S. Pat. No. 6,355,271.
[41] WO00/23105.
[42] WO01/22972.
[43] Kandimalla et al. (2003) Nucleic Acids Research 31:2393-2400.
[44] WO02/26757.
[45] WO99/62923.
[46] Krieg (2003) Nature Medicine 9:831-835.
[47] McCluskie et al. (2002) FEMS Immunology and Medical Microbiology 32:179-185.
[48] WO98/40100.
[49] U.S. Pat. No. 6,207,646.
[50] U.S. Pat. No. 6,239,116.
[51] U.S. Pat. No. 6,429,199.
[52] Kandimalla et al. (2003) Biochemical Society Transactions 31 (part 3):654-658.
[53] Blackwell et al. (2003) J Immunol 170:4061-4068.
[54] Krieg (2002) Trends Immunol 23:64-65.
[55] WO01/95935.
[56] Kandimalla et al. (2003) BBRC 306:948-953.
[57] Bhagat et al. (2003) BBRC 300:853-861.
[58] WO03/035836.
[59] Myers et al. (1990) pages 145-156 of Cellular and molecular aspects of endotoxin reactions.
[60] Ulrich (2000) Chapter 16 (pages 273-282) of reference 119.
[61] Johnson et al. (1999) J Med Chem 42:4640-9.
[62] Baldrick et al. (2002) Regulatory Toxicol Pharmacol 35:398-413.
[63] GB-A-2220211.
[64] U.S. Pat. No. 4,680,338.
[65] U.S. Pat. No. 4,988,815.
[66] WO92/15582.
[67] Stanley (2002) Clin Exp Dermatol 27:571-577.
[68] Wu et al. (2004) Antiviral Res. 64(2):79-83.
[69] Vasilakos et al. (2000) Cell Immunol. 204(1):64-74.
[70] U.S. Pat. Nos. 4,689,338, 4,929,624, 5,238,944, 5,266,575, 5,268,376, 5,346,905, 5,352,784, 5,389,640, 5,395,937, 5,482,936, 5,494,916, 5,525,612, 6,083,505, 6,440,992, 6,627,640, 6,656,938, 6,660,735, 6,660,747, 6,664,260, 6,664,264, 6,664,265, 6,667,312, 6,670,372, 6,677,347, 6,677,348, 6,677,349, 6,683,088, 6,703,402, 6,743,920, 6,800,624, 6,809,203, 6,888,000 and 6,924,293.
[71] Jones (2003) Curr Opin Investig Drugs 4:214-218.
[72] WO2004/060308.
[73] WO2004/064759.
[74] U.S. Pat. No. 6,924,271.
[75] US2005/0070556.
[76] U.S. Pat. No. 5,658,731.
[77] U.S. Pat. No. 5,011,828.
[78] WO2004/87153.
[79] U.S. Pat. No. 6,605,617.
[80] WO02/18383.
[81] WO2004/018455.
[82] WO03/082272.
[83] WO2006/002422.
[84] Johnson et al. (1999) Bioorg Med Chem Lett 9:2273-2278.
[85] Evans et al. (2003) Expert Rev Vaccines 2:219-229.
[86] Andrianov et al. (1998) Biomaterials 19:109-115.
[87] Payne et al. (1998) Adv Drug Delivery Review 31:185-196.
[88] U.S. Pat. No. 5,057,540.
[89] WO96/33739.
[90] EP-A-0109942.
[91] WO96/11711.
[92] WO00/07621.
[93] Barr et al. (1998) Advanced Drug Delivery Reviews 32:247-271.
[94] Sjolanderet et al. (1998) Advanced Drug Delivery Reviews 32:321-338.
[95] Pizza et al. (2000) Int J Med Microbiol 290:455-461.
[96] WO95/17211.
[97] WO98/42375.
[98] Singh et al] (2001) J Cont Release 70:267-276.
[99] WO99/27960.
[100] U.S. Pat. No. 6,090,406
[101] U.S. Pat. No. 5,916,588
[102] EP-A-0626169.
[103] WO99/52549.
[104] WO01/21207.
[105] WO01/21152.
[106] Signorelli & Hadden (2003) Int Immunopharmacol 3(8):1177-86.
[107] WO2004/064715.
[108] Cooper (1995) Pharm Biotechnol 6:559-80.
[109] WO03/011223.
[110] Meraldi et al. (2003) Vaccine 21:2485-2491.
[111] Pajak et al. (2003) Vaccine 21:836-842.
[112] U.S. Pat. No. 6,586,409.
[113] Wong et al. (2003) J Clin Pharmacol 43(7):735-42.
[114] US2005/0215517.
[115] WO90/14837.
[116] Podda & Del Giudice (2003) Expert Rev Vaccines 2:197-203.
[117] Podda (2001) Vaccine 19: 2673-2680.
[118] Vaccine Design: The Subunit and Adjuvant Approach (eds. Powell & Newman) Plenum Press 1995 (ISBN 0-30644867-X).
[119] Vaccine Adjuvants: Preparation Methods and Research Protocols (Volume 42 of Methods in Molecular Medicine series). ISBN: 1-59259-083-7. Ed. O'Hagan.
[120] Allison & Byars (1992) Res Immunol 143:519-25.
[121] Hariharan et al. (1995) Cancer Res 55:3486-9.
[122] WO95/11700.
[123] U.S. Pat. No. 6,080,725.
[124] WO2005/097181.
[125] WO2005/089837.
[126] U.S. Pat. No. 6,692,468.
[127] WO00/07647.
[129] U.S. Pat. No. 5,971,953.
[130] U.S. Pat. No. 4,060,082.
[131] EP-A-0520618.
[132] WO98/01174.
[133] Bodanszky (1993) Principles of Peptide Synthesis (ISBN: 0387564314).
[134] Fields et al. (1997) Meth Enzymol 289: Solid-Phase Peptide Synthesis. ISBN: 0121821900.
[135] Chan & White (2000) Fmoc Solid Phase Peptide Synthesis. ISBN: 0199637245.
[136] Kullmann (1987) Enzymatic Peptide Synthesis. ISBN: 0849368413.
[137] Ibba (1996) Biotechnol Genet Eng Rev 13:197-216.
[138] Srivastava et al. (2002) J Virol 76:2835-47.
[139] Srivastava et al. (2003) J Virol 77:11244-59.

What is claimed is:

1. A complex comprising a HIV Env polypeptide and a HIV Tat polypeptide, wherein (i) the Env and Tat polypeptides are covalently linked, and (ii) the complex can bind specifically to CD4.

2. The complex of claim 1, wherein the Env and Tat are from HIV-1.

3. The complex of claim 2, wherein the Env and Tat are from HIV-1 group M.

4. The complex of claim 3, wherein the Env and Tat are from a subtype B strain.

5. The complex of claim 3, wherein the Env and Tat are from a subtype C strain.

6. The complex of claim 1, wherein the Env and Tat are linked via a homobifunctional cross linker.

7. The complex of claim 1, wherein the Env and Tat are linked via reaction with formaldehyde or a dialdehyde.

8. The complex of claim 1, wherein the Env and Tat are present at essentially a 1:1 molar ratio.

9. A method for preparing a complex that comprises a HIV Env polypeptide and a HIV Tat polypeptide, comprising the step of allowing Env and Tat polypeptides to interact under reaction conditions where they become covalently linked to each other without removing the Env protein's ability to bind specifically to CD4.

10. The method of claim 9, wherein the Env and Tat are from HIV-1.

11. The method of claim 10, wherein the Env and Tat are from HIV-1 group M.

12. The method of claim 11, wherein the Env and Tat are from a subtype B strain.

13. The method of claim 11, wherein the Env and Tat are from a subtype C strain.

14. The method of claim 9, wherein the Env and Tat are linked via a homobifunctional cross linker.

15. The method of claim 9, wherein the Env and Tat are linked via reaction with formaldehyde or a dialdehyde.

16. The method of claim 9, wherein the Env and Tat are present at essentially a 1:1 molar ratio.


SEQUENCE LISTING (12 sequences)

SEQ ID NO: 1 - protein, 856 aa, Human immunodeficiency virus HIV-1
MRVKEKYQHLWRWGWRWGTMLLGMLMICSATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 2 - protein, 483 aa, Human immunodeficiency virus HIV-1
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 3 - protein, 345 aa, Human immunodeficiency virus HIV-1
AVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDKWASLWNWFNITNWLWYIKLFIMIVGGLVGLRIVFAVLSIVNRVRQGYSPLSFQTHLPTPRGPDRPEGIEEEGGERDRDRSIRLVNGSLALIWDDLRSLCLFSYHRLRDLLLIVTRIVELLGRRGWEALKYWWNLLQYWSQELKNSAVSLLNATAIAVAEGTDRVIEVVQGACRAIRHIPRRIRQGLERILL

SEQ ID NO: 4 - protein, 852 aa, Human immunodeficiency virus HIV-2
  Xaa 739: Xaa is any amino acid
MCGKSLLCVASLLASAYLVYCTQYVTVFYGVPVWRNASIPLFCATKNRDTWGTIQCKPDNDDYQEITLNVTEAFDAWDNTVTEQAVEDVWSLFETSIKPCVKLTPLCVAMSCNSTTNNTTTTGSTTGMSEINETSPSYSDNCTGLGKEEIVNCQFYMTGLERDKKKQYNETWYSKDVVCESNNTKDGKNRCYMNHCNTSVITESCDKHYWDAIKFRYCAPPGYALLRCNDTNYSGFEPKCSKVVASTCTRMMETQTSTWFGFNGTRAENRTYIYWHGRDNRTIISLNKYYNLSIHCKRPGNKTVVPITLMSGLVFHSQPINTRPRQAWCWFKGKWREAMQEVKQTLIKHPRYKGTNDTKNINFTKPGRGSDPEVAYMWTNCRGEFLYCNMTWFLNWVENRPNQTQHNYAPCHIRQIINTWHKVGKNVYLPPREGQLTCNSTVTSIIANIDVNSNQTNITFSAEVAELYRLELGDYKLIEVTPIGFAPTREKRYSSAPVRNKRGVFVLGFLGFLATAGSAMGAASLTLSAQSRTLLAGIVQQQQQLLDVVKRQQEMLRLTVWGTKNLQARVTAIEKYLKDQAQLNSWGCAFRQVCHTTVPWVNDSLSPDWNNMTWQEWEKQVRYLEANISQSLEQAQIQQEKNMYELQKLNSWDVFGNWFDLTSWIKYIQYGVYIVVGVIVLRIAIYIVQLLSRLRKGYRPVFSSPPGYLQQIHIHTDRGQPANEGTEEDDRDDDGYDLXPWPINYIHFLIHLLTRLLTGLYKICRDLLSTNSPTHRLISQNLTAIRDWLRLKAAYLQYGGEWIQEAFQAFAKTTRETLASAWGGLCAAVQRVGRGILAVPRRIRQGAEIALL

SEQ ID NO: 5 - protein, 483 aa, Human immunodeficiency virus HIV-2
YCTQYVTVFYGVPVWRNASIPLFCATKNRDTWGTIQCKPDNDDYQEITLNVTEAFDAWDNTVTEQAVEDVWSLFETSIKPCVKLTPLCVAMSCNSTTNNTTTTGSTTGMSEINETSPSYSDNCTGLGKEEIVNCQFYMTGLERDKKKQYNETWYSKDVVCESNNTKDGKNRCYMNHCNTSVITESCDKHYWDAIKFRYCAPPGYALLRCNDTNYSGFEPKCSKVVASTCTRMMETQTSTWFGFNGTRAENRTYIYWHGRDNRTIISLNKYYNLSIHCKRPGNKTVVPITLMSGLVFHSQPINTRPRQAWCWFKGKWREAMQEVKQTLIKHPRYKGTNDTKNINFTKPGRGSDPEVAYMWTNCRGEFLYCNMTWFLNWVENRPNQTQHNYAPCHIRQIINTWHKVGKNVYLPPREGQLTCNSTVTSIIANIDVNSNQTNITFSAEVAELYRLELGDYKLIEVTPIGFAPTREKRYSSAPVRNKR

SEQ ID NO: 6 - protein, 350 aa, Human immunodeficiency virus HIV-2
  Xaa 237: Xaa is any amino acid
GVFVLGFLGFLATAGSAMGAASLTLSAQSRTLLAGIVQQQQQLLDVVKRQQEMLRLTVWGTKNLQARVTAIEKYLKDQAQLNSWGCAFRQVCHTTVPWVNDSLSPDWNNMTWQEWEKQVRYLEANISQSLEQAQIQQEKNMYELQKLNSWDVFGNWFDLTSWIKYIQYGVYIVVGVIVLRIAIYIVQLLSRLRKGYRPVFSSPPGYLQQIHIHTDRGQPANEGTEEDDRDDDGYDLXPWPINYIHFLIHLLTRLLTGLYKICRDLLSTNSPTHRLISQNLTAIRDWLRLKAAYLQYGGEWIQEAFQAFAKTTRETLASAWGGLCAAVQRVGRGILAVPRRIRQGAEIALL

SEQ ID NO: 7 - protein, 637 aa, Human immunodeficiency virus HIV-1
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCSFNISTSIRGKVQKEYAFFYKLDIIPIDNDTTSYKLTSCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKRAVGIGALFLGFLGAAGSTMGAASMTLTVQARQLLSGIVQQQNNLLRAIEAQQHLLQLTVWGIKQLQARILAVERYLKDQQLLGIWGCSGKLICTTAVPWNASWSNKSLEQIWNHTTWMEWDREINNYTSLIHSLIEESQNQQEKNEQELLELDK

SEQ ID NO: 8 - protein, 446 aa, Human immunodeficiency virus HIV-1
  Xaa 130: Xaa represents a mutant V2 loop with between 4 and 15 amino acids
SATEKLWVTVYYGVPVWKEATTTLFCASDAKAYDTEVHNVWATHACVPTDPNPQEVVLVNVTENFNMWKNDMVEQMHEDIISLWDQSLKPCVKLTPLCVSLKCTDLKNDTNTNSSSGRMIMEKGEIKNCXCNTSVITQACPKVSFEPIPIHYCAPAGFAILKCNNKTFNGTGPCTNVSTVQCTHGIRPVVSTQLLLNGSLAEEEVVIRSVNFTDNAKTIIVQLNTSVEINCTRPNNNTRKRIRIQRGPGRAFVTIGKIGNMRQAHCNISRAKWNNTLKQIASKLREQFGNNKTIIFKQSSGGDPEIVTHSFNCGGEFFYCNSTQLFNSTWFNSTWSTEGSNNTEGSDTITLPCRIKQIINMWQKVGKAMYAPPISGQIRCSSNITGLLLTRDGGNSNNESEIFRPGGGDMRDNWRSELYKYKVVKIEPLGVAPTKAKRRVVQREKR

SEQ ID NO: 9 - protein, 619 aa, Human immunodeficiency virus HIV-1
SAVEKLWVTVYYGVPVWKEATTTLFCASDAKAYDTEVHNVWATHACVPTDPNPQEIVLENVTENFNMWKNNMVEQMHEDIISLWDQSLKPCVKLTPLCVTLHCTNLKNATNTKSSNWKEMDRGEIKNCSFKVGAGKLINCNTSVITQACPKVSFEPIPIHYCAPAGFAILKCNDKKFNGSGPCTNVSTVQCTHGIRPVVSTQLLLNGSLAEEGVVIRSENFTDNAKTIIVQLKESVEINCTRPNNNTRKSITIGPGRAFYATGDIIGDIRQAHCNISGEKWNNTLKQIVTKLQAQFGNKTIVFKQSSGGDPEIVMHSFNCGGEFFYCNSTQLFNSTWNNTIGPNNTNGTITLPCRIKQIINRWQEVGKAMYAPPIRGQIRCSSNITGLLLTRDGGKEISNTTEIFRPGGGDMRDNWRSELYKYKVVKIEPLGVAPTKAISSVVQSEKSAVTLGAMFLGFLGAAGSTMGARSLTLTVQARQLLSGIVQQQNNLLRAIEAQQHLLQLTVWGIKQLQARVLAVERYLKDQQLLGIWGCSGKLICTTAVPWNASWSNKSLDQIWNNMTWMEWEREIDNYTNLIYTLIEESQNQQEKNEQELLELDKWASLWNWFDISKWLWYI

SEQ ID NO: 10 - protein, 86 aa, Human immunodeficiency virus HIV-1
MEPVDPRLEPWKHPGSQPKTACTNCYCKKCCFHCQVCFITKALGISYGRKKRRQRRRAHQNSQTHQASLSKQPTSQPRGDPTGPKE

SEQ ID NO: 11 - protein, 130 aa, Human immunodeficiency virus HIV-2
METPLKAPESSLMSYNEPSSCTSERDVGSQELAKQGEELLSQLHRPLEPCNNKCYCKGCCFHCQLCFLNKGLGICYDRKGRRRRTPKKTKAHSSSASDKSISTRTGNSQPEKKQKKTLETTLETARGLGR

SEQ ID NO: 12 - protein, 86 aa, Human immunodeficiency virus HIV-1
MEPVDPRLEPWKHPGSQPKTACTNCYCKKCCFHCQVCFITKALGISYGRKKRRQRRRPPQGSQTHQVSLSKQPTSQSRGDPTGPKE